(19) United States
(12) Patent Application Publication
(10) Pub. No.: US 2025/0353816 A1
(43) Pub. Date: Nov. 20, 2025

(54) QUINOLINES AS MODULATORS OF POLRMT

(71) Applicant: Pretzel Therapeutics, Inc., Waltham, MA (US)

(72) Inventors: Andrew Mark Griffin, Quebec (CA); Paul S. Charifson, Carrabassett Valley, ME (US); Jeremy Green, Waltham, MA (US); Gabriel Martinez Botella, Wayland, MA (US)

(73) Assignee: Pretzel Therapeutics, Inc., Waltham, MA (US)

(21) Appl. No.: 19/207,820

(22) Filed: May 14, 2025

Related U.S. Application Data
(60) Provisional application No. 63/647,907, filed on May 15, 2024.

(51) Int. Cl. C07D 215/48; C07D 215/12; C07D 215/14; C07D 215/38; C07D 401/06

(52) U.S. Cl. CPC C07D 215/48; C07D 215/12; C07D 215/14; C07D 215/38; C07D 401/06

(57) ABSTRACT

The present invention provides novel quinoline compounds that are inhibitors of mitochondrial RNA polymerase for treating various diseases such as cancer and others associated with metabolic disorders and mitochondrial dysfunction.

### SEQUENCE LISTING

The instant application contains a Sequence Listing which has been submitted electronically in XML format and is hereby incorporated by reference in its entirety. Said XML copy, created on Jul. 28, 2025, is named A748-7_8E_SL.xml and is 3,328 bytes in size.

### FIELD OF THE INVENTION

The present invention relates to novel POLRMT modulators, their prodrugs, their pharmaceutically acceptable salts, and pharmaceutical compositions thereof. The present invention also relates to methods of using such compounds and compositions, including to inhibit or promote POLRMT, and to treat various neurodegenerative and metabolic disorders, cancer, and also disorders related to aging and mitochondrial diseases.

### BACKGROUND OF THE INVENTION

Human mitochondrial RNA polymerase, POLRMT (also referred to as h-mtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368). See, e.g., Arnold, J. J., et al., “Human mitochondrial RNA polymerase: Structure-function, mechanism and inhibition,” Biochim. Biophys. Acta, 1819, 948-960 (2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Regarding the POLRMT NTD, structurally it resembles the N-terminal domain (also called the promoter-binding domain) of T7 RNA polymerase. However, for promoter-specific transcription initiation POLRMT requires assistance from additional transcription factors, whereas T7 RNA polymerase does not.

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site. See, e.g., Basu, U., et al., “Structure, mechanism, and regulation of mitochondrial DNA transcription initiation,” J. Biol. Chem., 295(52), 18406-425 (2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id.

The mitochondrial genome encodes the various subunits of the electron transport chain. See, e.g., Shokolenko, I. N., et al., “Maintenance and expression of mammalian mitochondrial DNA,” Annu. Rev. Biochem., 85, 133-160 (2016). Specifically, transcription of the mitochondrial genome is necessary for the expression of 13 subunits of the oxidative phosphorylation (OXPHOS) system, as well as two rRNAs and 22 tRNAs. See, e.g., Shokolenko, I. N., et al., “Mitochondrial transcription in mammalian cells,” Frontiers in Bioscience, Landmark, 22, 835-853 (2017). Thus, POLRMT is essential for biogenesis of the OXPHOS system, resulting in ATP production. This, in turn, is vital for energy homeostasis in the cell.

Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer. See Siegel, R. L. et al., “Cancer statistics 2022.” CA Cancer J. Clin. (72) 7-33 (2022). High rates of OXPHOS have been shown to support growth in cancer cell lines, including in a subset of diffuse large B cell lymphoma cells. See, e.g., DeBeradinis, R. J., “A mitochondrial power play in lymphoma,” Cancer Cell, 22, 423-24 (2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity. Rodrigues, M. F., et al., “Enhanced OXPHOS, glutaminolysis and β-oxidation constitute the metastatic phenotype of melanoma cells,” Biochem. J. 473: 703-715 (2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown AML cells demonstrated a reduction in POLRMT levels, decreased oxidative phosphorylation, and increased cell death as compared to control AML cells. See Bralha, F. N., et al., “Targeting mitochondrial RNA polymerase in acute myeloid leukemia,” Oncotarget, 6(35), 37216-228 (2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene. Salem, A. F., et al. “Mitochondrial biogenesis in epithelial cancer cells promotes breast cancer tumor growth and confers autophagy resistance,” Cell Cycle, 11(22), 4174-80 (2012). Recently, the expression of POLRMT in non-small cell lung cancer (NSCLC) has been examined. See Zhou, T. et al., “The requirement of mitochondrial RNA polymerase for non-small cell lung cancer cell growth,” Cell Death and Disease, 12, 751 (2021). While POLRMT mRNA and protein were detected in normal human lung tissue, their levels were significantly higher in cancer tissue. Similar results were obtained when comparing primary lung epithelial cells to NSCLC cells. Using short hairpin RNA (shRNA) to silence POLRMT mRNA and downregulate POLRMT protein resulted in inhibition of NSCLC cell viability, proliferation, migration, and invasion. Moreover, silencing of POLRMT significantly induced apoptosis activation in both primary and established NSCLC cells. Injection of POLRMT shRNA in an adeno-associated virus construct into tumors effectively inhibited NSCLC xenograft growth in mice. Taken together, these data suggest that POLRMT could be an oncogenic gene for NSCLC.

The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy. See Yu, H.-J., “Targeting mitochondrial metabolism and RNA polymerase POLRMT to overcome multidrug resistance in cancer,” Front. Chem., 9:775226 (2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small-molecule inhibitors of POLRMT have been developed. See, e.g., EP 3 598 972 A1; WO 2019/057821 A1; and WO 2020/188049 A1. Some of these inhibitors have been shown to be useful in inhibiting cancer cell proliferation without affecting control cells. See Bonekamp, N. A., el al., “Small-molecule inhibitors of human mitochondrial DNA transcription,” Nature, 588, 712-716 (2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals. Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated. See Mennuni, M. et al., “Metabolic resistance to the inhibition of mitochondrial transcription revealed by CRISPER-Cas9 screen,” EMBO reports, 23: e53054 1-18 (2022). Using a CRISPR-Cas9 whole-genome screen, loss of genes belonging to von Hippel-Lindau (VHL) and mammalian target of rapamycin complex I (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity. See Pospisilik, J. A., et al., “Targeted deletion of AIF decreases mitochondrial oxidative phosphorylation and protects from obesity and diabetes,” Cell, 131, 476-91 (2007). Correlated with these phenotypic changes were the metabolic alterations of increased glucose uptake and enhanced fuel utilization. Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component. See, e.g., Filograna, R., et al., “Mitochondrial DNA copy number in human disease: the more the better?” FEBS Letters, 595, 976-1002 (2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POLy) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurogenerative disorders, cancer, and aging. See e.g., Filograna, R., et al., “Mitochondrial DNA copy number in human disease: the more the better?” FEBS Letters, 595, 976-1002 (2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease. See Olahova, M., et al., “POLRMT mutations impair mitochondrial transcription causing neurological disease.” Nat. Commun., 12, 1135 (2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Pharmacological inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat various neurodegenerative and metabolic disorders, cancer, and also disorders related to aging and mitochondrial diseases. The compounds and their pharmaceutically acceptable salts are particularly useful as modulators of POLRMT.

In one embodiment, the present invention is directed to a compound, a prodrug thereof, or a pharmaceutically acceptable salt thereof, represented by formula (I):

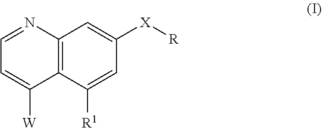

wherein:
X is —C₁-C₆ alkyl-, —N(R³)—, —C(O)—;
W is C₆-C₁₀ aryl, C₆-C₁₀ cycloaryl, or 5-10 membered heteroaryl, wherein the aryl, cycloaryl, or heteroaryl are each optionally substituted with one or more groups independently selected from the group consisting of fluoro, chloro, C₁-C₄ alkyl, trifluoromethyl, difluoromethyl, cyano, hydroxyl, and C₁-C₄ alkoxyl;
R is hydrogen, C₁-C₆ alkyl, hydroxy, NR²R³, acyl, carboxy, C(O)NR²R³, or NR⁴—S(O)₂R² wherein the alkyl group is optionally substituted with one or more groups each independently selected from the group consisting of hydroxyl, cyano, fluoro, C₁-C₄ alkoxyl, C₁-C₄ haloalkoxyl, aryl, 5- or 6-membered heterocyclyl, C(O)OR⁴, NR²R³, and C(O)NR²R³,
or R is C₁-C₄ alkoxyl optionally substituted with hydroxyl, fluoro, NR²R³, carboxyl, and C(O)NR²R³,
or R is C₃-C₆ cycloalkyl, or 5- or 6-membered heterocyclyl, wherein the cycloalkyl and heterocyclyl are each optionally substituted with oxo or C₁-C₄ alkyl,
or R is C₆-C₁₀ aryl or C₅-C₆ heteroaryl containing one or more heteroatoms that is N, O, or S, wherein the aryl and heteroaryl are optionally substituted with one or more groups each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and acyl;
R¹ is hydrogen, fluoro, chloro, hydroxyl, cyano, or C₁-C₃ alkyl optionally substituted with one or more fluorines, C₃-C₄ cycloalkyl, C₁-C₂ alkoxyl or C₁-C₂ haloalkoxyl;
each R² is independently hydrogen, C₁-C₄ alkyl, C₃-C₆ cycloalkyl, hydroxyl, or aryl, wherein the alkyl, cycloalkyl, and aryl are optionally substituted with one or more groups selected from the group consisting of C₃-C₆ cycloalkyl, fluoro, hydroxyl, C₁-C₄ alkoxyl, C₁-C₄ haloalkoxyl, aryl, carboxyl, C(O)NR⁴R⁴;
each R³ is independently R², C(O)C₁-C₄ alkyl, C(O)C₃-C₆ cycloalkyl, C(O)C₆-C₁₀ aryl, C(O)-5- to 10-membered heteroaryl, C(O)O—C₁-C₄ alkyl, or C(O)NHC₁-C₄ alkyl, wherein each alkyl, cycloalkyl, aryl, and heteroaryl are optionally substituted with one or more groups selected from the group consisting of C₁-C₄ alkyl, fluoro, chloro, trifluoromethyl, difluoromethyl, cyano, hydroxyl, and C₁-C₄ alkoxyl,
or if R² and R³ are attached to the same nitrogen atom, R² and R³ together with their connecting nitrogen can form a 5- or 6-membered heterocyclic ring optionally containing another heteroatom that is N, O, or S, and optionally substituted with one or two groups each independently selected from the group consisting of fluoro, chloro, C₁-C₄ alkyl, C₁-C₄ alkoxyl, C₁-C₄ haloalkoxyl, acyl, carboxy, C₁-C₄ alkyl carboxy, and C₁-C₄ alkylcarboxylate; and
R⁴ is hydrogen or C₁-C₄ alkyl.

A further embodiment of the present invention are compounds of the invention (that is, compounds of formula (I), their pharmaceutically acceptable salts, or prodrugs of the compounds wherein one or more hydrogen is substituted with a deuterium atom.

Further embodiments of the invention are pharmaceutical compositions comprising a compound of the invention, a pharmaceutically acceptable salt thereof, or a prodrug thereof and one or more pharmaceutically acceptable excipients.

Further embodiments of the invention are methods of treating a disease, such methods comprising administering to a subject in need thereof a therapeutically effective amount of a compound of the invention, a prodrug thereof, or a pharmaceutically acceptable salt thereof. In some embodiments, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, choriocarcinoma, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, polycythemia vera, prostate cancer, rhabdomyosarcoma, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, non-alcoholic steatohepatitis (NASH), and related metabolic syndromes such as non-alcoholic fatty liver disease (NAFLD). In some embodiments, the disease is related to aging or a mitochondrial disorder.

Additional embodiments of the invention are methods of treating neurodegenerative disorders and metabolic disorders, such as those identified in Bonekamp, N. A. et al. “Small-molecule inhibitors of human mitochondrial DNA transcription,” Nature, 588, 712-716 (2020), Filograna, R. et al, “Mitochondrial DNA copy number in human disease: the more the better?” FEBS Lett., 595, 976-1002 (2021), Wrendenber, A. et al. “Respiratory chain dysfunction in skeletal muscle does not cause insulin resistance,” Biochem. Biophys. Res. Comm., 350, 202-207 (2006), Pospililik, J. A. et al. “Targeted deletion of AIF decreases mitochondrial oxidative phosphorylation and protects from obesity and diabetes,” Cell, 131, 476-491 (2007), and PCT Published International Publication No. WO 2019/057821 A1 and references therein.

Further embodiments of the invention are methods of treating disease of aging.

### DETAILED DESCRIPTION OF THE INVENTION

Modulators of POLRMT are useful in compositions and methods suitable for treating many disorders, such as cancer, neurodegenerative disorders, metabolic disorders, as well as diseases related to aging and mitochondrial diseases. Provided herein are compounds of formula (I), pharmaceutically acceptable salts thereof, prodrugs thereof, and pharmaceutical compositions comprising such compounds, their salts, or their prodrugs that are useful in treating a condition or disease, such as cancer, neurodegenerative disorders, and metabolic disorders.

### Definitions

The term “alkyl” as used herein refers to both branched- and straight-chain saturated aliphatic hydrocarbon groups having the specified number of carbon atoms in a specified range. For example the term “C₁-C₆ alkyl” means linear or branched chain alkyl groups, including all possible isomers, having 1, 2, 3, 4, 5, or 6 carbon atoms. Furthermore, alkyl groups allow for substituents to be located on any of the carbon atoms. For example, a substituted C₃ alkyl group allows for the substituent to be located on any of the three carbon atoms.

The term “alkoxy” or “alkoxyl” as used herein refers to an —O-alkyl group. For example, the term “C₁-C₄ alkoxyl” means —O—C₁-C₄ alkyl. Examples of alkoxyl include methoxyl, ethoxyl, propoxyl (e.g., n-propoxyl and isopropoxyl), and the like.

The term “haloalkoxy” or “haloalkoxyl” as used herein refers to an —O-alkyl group in which at least one of the hydrogen atoms of the alkyl group is replaced with a halogen atom. Examples of haloalkoxyl include trifluoromethoxyl, 2,2,2-trifluoroethoxyl, and the like.

The term “alkanoyl” or “acyl” as used herein refers to an —C(O)-alkyl group. For example, the term “C₁-C₆ alkanoyl” means —C(O)—C₁-C₆ alkyl. Examples of alkanoyl include acetyl, propionyl, butyryl, and the like.

The term “cycloalkyl” as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term “aryl” as used herein refers to a monocyclic or fused bicyclic ring system having the characteristics of aromaticity, wherein at least one ring contains a completely conjugated pi-electron system. Typically, aryl groups contain 6 to 14 carbon atoms (“C₆-C₁₄ aryl”) or preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom of the aromatic portion or a C or N atom of the non-aromatic portion of the ring system. Examples, without limitation, of aryl groups include phenyl, biphenyl, naphthyl, anthracenyl, indanyl, indenyl, and tetrahydronaphthyl.

The term “cycloaryl” herein refers to a polycyclic group wherein an aryl group is fused to a 5- or 6-membered aliphatic ring. For example, “C₆-C₁₂ cycloaryl” means a C₆-C₁₂ aryl fused to a 5- or 6-membered aliphatic ring.

The term “heteroaryl” as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatoms, at least one ring is aromatic, each N is optionally in the form of an oxide, and each S in a ring which is not aromatic is optionally S(O) or S(O)₂. Typically, heteroaryl groups contain 5 to 14 ring atoms (“5-14 membered heteroaryl”), and preferably 5 to 12 ring atoms (“5- to 12-membered heteroaryl”). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term “heteroaryloxy” or “heteroaryloxyl” as used herein refers to an —O— heteroaryl group.

The term “oxo” as used herein refers to a group which consists of oxygen which is double bonded to carbon or any other element.

The term “heterocycle”, “heterocyclyl”, or “heterocyclic” as used herein represents a stable 3- to 10-membered monocyclic, non-aromatic ring that is either saturated or unsaturated, and that consists of carbon atoms and from one to two heteroatoms selected from the group consisting of N, O, and S. Examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, and oxazepanyl.

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” means that the groups may be the same or different.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen”, the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (T Higuchi and W Stella) and “Bioreversible Carriers in Drug Design,” Pergamon Press, 1987 (ed. E B Roche, American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety. Some non-limiting examples of prodrugs in accordance with the invention include: (i) where the compound contains a carboxylic acid functionality —(COOH), an ester thereof, for example, replacement of the hydrogen with (C₁-C₆)alkyl; (ii) where the compound contains an alcohol functionality (—OH), an ether thereof, for example, replacement of the hydrogen with (C₁-C₆)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₆ alkanoyl. Further examples of replacement groups in accordance with the foregoing examples and examples of other prodrug types may be found in the aforementioned references.

The terms “treatment”, “treating” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

### Compounds

In one embodiment, the present invention is directed to a compound, a prodrug thereof, or a pharmaceutically acceptable salt thereof, represented by formula (I):

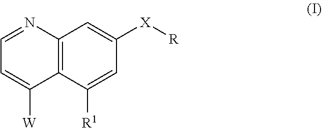

wherein:
X is —C₁-C₆ alkyl-, —N(R³)—, —C(O)—;
W is C₆-C₁₀ aryl, C₆-C₁₀ cycloaryl, or 5-10 membered heteroaryl, wherein the aryl, cycloaryl, or heteroaryl are each optionally substituted with one or more groups independently selected from the group consisting of fluoro, chloro, C₁-C₄ alkyl, trifluoromethyl, difluoromethyl, cyano, hydroxyl, and C₁-C₄ alkoxyl;
R is hydrogen, C₁-C₆ alkyl, hydroxy, NR²R³, acyl, carboxy, C(O)NR²R³, or NR⁴—S(O)₂R² wherein the alkyl group is optionally substituted with one or more groups each independently selected from the group consisting of hydroxyl, cyano, fluoro, C₁-C₄ alkoxyl, C₁-C₄ haloalkoxyl, aryl, 5- or 6-membered heterocyclyl, C(O)OR⁴, NR²R³, and C(O)NR²R³,
or R is C₁-C₄ alkoxyl optionally substituted with hydroxyl, fluoro, NR²R³, carboxyl, and C(O)NR²R³,
or R is C₃-C₆ cycloalkyl, or 5- or 6-membered heterocyclyl, wherein the cycloalkyl and heterocyclyl are each optionally substituted with oxo or C₁-C₄ alkyl,
or R is C₆-C₁₀ aryl or C₅-C₆ heteroaryl containing one or more heteroatoms that is N, O, or S, wherein the aryl and heteroaryl are optionally substituted with one or more groups each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and acyl;
R¹ is hydrogen, fluoro, chloro, hydroxyl, cyano, or C₁-C₃ alkyl optionally substituted with one or more fluorines, C₃-C₄ cycloalkyl, C₁-C₂ alkoxyl or C₁-C₂ haloalkoxyl;
each R² is independently hydrogen, C₁-C₄ alkyl, C₃-C₆ cycloalkyl, hydroxyl, or aryl, wherein the alkyl, cycloalkyl, and aryl are optionally substituted with one or more groups selected from the group consisting of C₃-C₆ cycloalkyl, fluoro, hydroxyl, C₁-C₄ alkoxyl, C₁-C₄ haloalkoxyl, aryl, carboxyl, C(O)NR⁴R⁴;
each R³ is independently R², C(O)C₁-C₄ alkyl, C(O)C₃-C₆ cycloalkyl, C(O)C₆-C₁₀ aryl, C(O)-5- to 10-membered heteroaryl, C(O)O—C₁-C₄ alkyl, or C(O)NHC₁-C₄ alkyl, wherein each alkyl, cycloalkyl, aryl, and heteroaryl are optionally substituted with one or more groups selected from the group consisting of C₁-C₄ alkyl, fluoro, chloro, trifluoromethyl, difluoromethyl, cyano, hydroxyl, and C₁-C₄ alkoxyl,
or if R² and R³ are attached to the same nitrogen atom, R² and R³ together with their connecting nitrogen can form a 5- or 6-membered heterocyclic ring optionally containing another heteroatom that is N, O, or S, and optionally substituted with one or two groups each independently selected from the group consisting of fluoro, chloro, C₁-C₄ alkyl, C₁-C₄ alkoxyl, C₁-C₄ haloalkoxyl, acyl, carboxy, C₁-C₄ alkyl carboxy, and C₁-C₄ alkylcarboxylate; and
R⁴ is hydrogen or C₁-C₄ alkyl.

In certain embodiments, W is phenyl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl.

In certain embodiments, W is phenyl optionally substituted with one or more groups, each independently selected from the group consisting of fluoro, chloro, C₁-C₄ alkyl, trifluoromethyl, difluoromethyl, cyano, hydroxyl, and C₁-C₄ alkoxyl, provided that at least one substituent is at an ortho position relative to the attachment point with the central ring.

In certain embodiments, W is 2-chloro-4-fluorophenyl.

In certain embodiments, W is 2-methyl-phenyl.

In certain embodiments, R¹ is hydrogen.

In certain embodiments, R¹ is methyl.

In certain embodiments, X is —C₁-C₆ alkyl-.

In certain embodiments, X is —N(R³)—.

In certain embodiments, X is —C(O)—.

In certain embodiments, X is —NH—.

In certain embodiments, X is —CH₂—.

In certain embodiments, R is hydrogen.

In certain embodiments, R is C₁-C₆ alkyl.

In certain embodiments, R is methyl.

In certain embodiments, R is n-propyl.

In certain embodiments, R is iso-propyl.

In certain embodiments, R is C₁-C₆ alkyl optionally substituted with one or more groups each independently selected from the group consisting of hydroxyl, cyano, fluoro, C₁-C₄ alkoxyl, C₁-C₄ haloalkoxyl, aryl, 5- or 6-membered heterocyclyl, C(O)OR⁴, NR²R³, and C(O)NR²R³.

In certain embodiments, R is methoxy.

In certain embodiments, R is 1-cyanoethyl.

In certain embodiments, R is 5- or 6-membered heterocyclyl.

In certain embodiments, R is morpholin-1-yl.

In certain embodiments, R is pyrrolidin-1-yl.

In certain embodiments, R is SO₂CH₃.

In certain embodiments, R is acyl.

In certain embodiments, R is NH₂.

In certain embodiments, R is NHCH₃.

In certain embodiments, R² is phenyl.

In certain embodiments, R² is OR¹.

In certain embodiments, R is CH₂CH₂OCH₃.

In certain embodiments, R is CH₂CH₂OH.

In certain embodiments, R² is C(O)NR¹R².

In certain embodiments, R² is C(O)NHCH₃.

In certain embodiments, the compound is (S)-1-((S)-3-(4-(2-chloro-4-fluorophenyl)quinolin-7-yl)-2-methylpropanoyl)piperidine-3-carboxylic acid, or a pharmaceutically acceptable salt thereof:

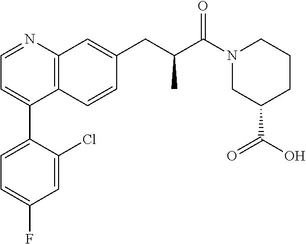 [SMILES: C[C@@H](Cc1ccc2c(-c3ccc(F)cc3Cl)ccnc2c1)C(=O)N1CCC[C@H](C(=O)O)C1]

In certain embodiments, the compound is (S)-1-((R)-3-(4-(2-chloro-4-fluorophenyl)quinolin-7-yl)-2-methylpropanoyl)piperidine-3-carboxylic acid, or a pharmaceutically acceptable salt thereof:

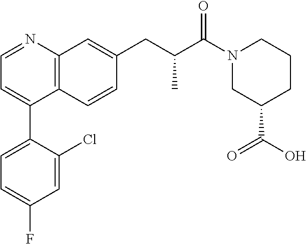 [SMILES: C[C@H](Cc1ccc2c(-c3ccc(F)cc3Cl)ccnc2c1)C(=O)N1CCC[C@H](C(=O)O)C1]

In certain embodiments, the compound is (S)-1-(N-(4-(2-chloro-4-fluorophenyl)quinolin-7-yl)-N-methyl-L-alanyl)piperidine-3-carboxylic acid, or a pharmaceutically acceptable salt thereof:

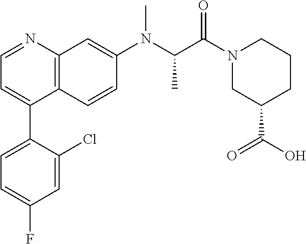 [SMILES: C[C@@H](C(=O)N1CCC[C@H](C(=O)O)C1)N(C)c1ccc2c(-c3ccc(F)cc3Cl)ccnc2c1]

In certain embodiments, the compound is (S)-1-(N-(4-(2-chloro-4-fluorophenyl)quinolin-7-yl)-N-methyl-D-alanyl)piperidine-3-carboxylic acid, or a pharmaceutically acceptable salt thereof:

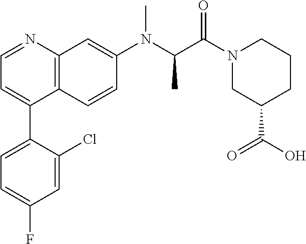 [SMILES: C[C@H](C(=O)N1CCC[C@H](C(=O)O)C1)N(C)c1ccc2c(-c3ccc(F)cc3Cl)ccnc2c1]

In certain embodiments, the compound is 2-((R)-1-((S)-3-(4-(2-chloro-4-fluorophenyl)quinolin-7-yl)-2-methylpropanoyl)piperidin-3-yl)acetic acid, or a pharmaceutically acceptable salt thereof:

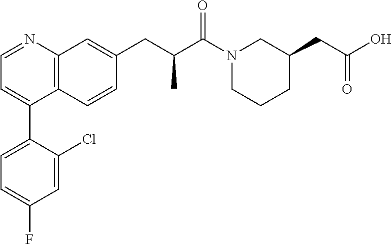 [SMILES: C[C@@H](Cc1ccc2c(-c3ccc(F)cc3Cl)ccnc2c1)C(=O)N1CCC[C@H](CC(=O)O)C1]

In certain embodiments, the compound is 2-((R)-1-((R)-3-(4-(2-chloro-4-fluorophenyl)quinolin-7-yl)-2-methylpropanoyl)piperidin-3-yl)acetic acid, or a pharmaceutically acceptable salt thereof:

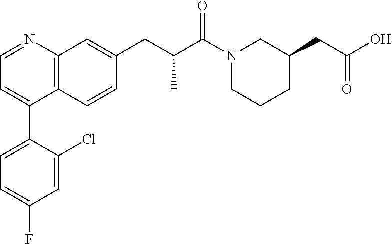 [SMILES: C[C@H](Cc1ccc2c(-c3ccc(F)cc3Cl)ccnc2c1)C(=O)N1CCC[C@H](CC(=O)O)C1]

In certain embodiments, the compound is 2-((R)-1-(N-(4-(2-chloro-4-fluorophenyl)quinolin-7-yl)-N-methyl-L-alanyl)piperidin-3-yl)acetic acid, or a pharmaceutically acceptable salt thereof:

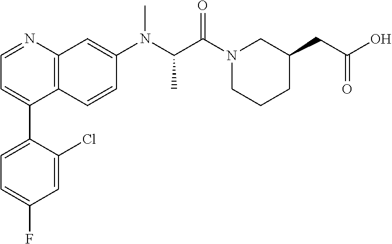 [SMILES: C[C@@H](C(=O)N1CCC[C@H](CC(=O)O)C1)N(C)c1ccc2c(-c3ccc(F)cc3Cl)ccnc2c1]

In certain embodiments, the compound is 2-((R)-1-(N-(4-(2-chloro-4-fluorophenyl)quinolin-7-yl)-N-methyl-D-alanyl)piperidin-3-yl)acetic acid, or a pharmaceutically acceptable salt thereof:

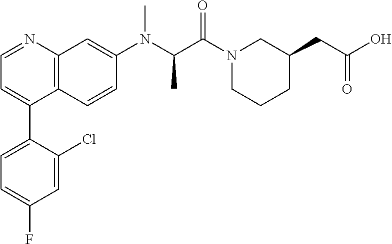 [SMILES: C[C@H](C(=O)N1CCC[C@H](CC(=O)O)C1)N(C)c1ccc2c(-c3ccc(F)cc3Cl)ccnc2c1]

In certain embodiments, the compound is N-methyl-2-(methyl(4-(o-tolyl)quinolin-7-yl)amino)acetamide, or a pharmaceutically acceptable salt thereof:

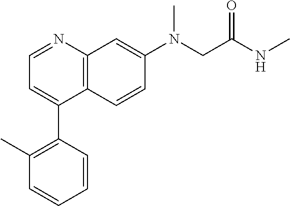

In certain embodiments, the compound is 2-methyl-2-(2-(methyl(4-(o-tolyl)quinolin-7-yl)amino)acetamido)propanoic acid, or a pharmaceutically acceptable salt thereof:

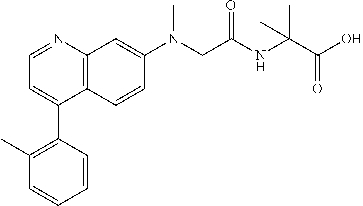 [SMILES: Cc1ccccc1-c1ccnc2cc(N(C)CC(=O)NC(C)(C)C(=O)O)ccc12]

In certain embodiments, the compound is N-methyl-N-(4-(o-tolyl)quinolin-7-yl)glycylglycine, or a pharmaceutically acceptable salt thereof:

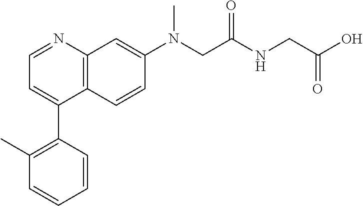 [SMILES: Cc1ccccc1-c1ccnc2cc(N(C)CC(=O)NCC(=O)O)ccc12]

In certain embodiments, the compound is N-(2-amino-2-oxoethyl)-2-(methyl(4-(o-tolyl)quinolin-7-yl)amino)acetamide, or a pharmaceutically acceptable salt thereof:

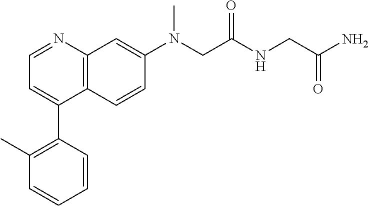 [SMILES: Cc1ccccc1-c1ccnc2cc(N(C)CC(=O)NCC(N)=O)ccc12]

In certain embodiments, the compound is N-(2-amino-2-oxoethyl)-N-methyl-2-(methyl(4-(o-tolyl)quinolin-7-yl)amino)acetamide, or a pharmaceutically acceptable salt thereof:

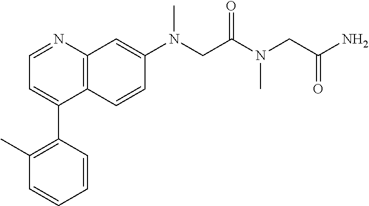 [SMILES: Cc1ccccc1-c1ccnc2cc(N(C)CC(=O)N(C)CC(N)=O)ccc12]

In certain embodiments, the compound is (S)-2-(2-(methyl(4-(o-tolyl)quinolin-7-yl)amino)acetamido)propanamide, or a pharmaceutically acceptable salt thereof:

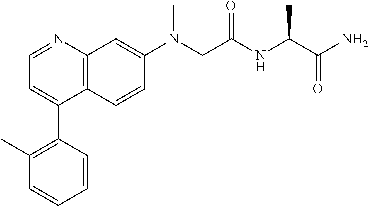 [SMILES: Cc1ccccc1-c1ccnc2cc(N(C)CC(=O)N[C@@H](C)C(N)=O)ccc12]

In certain embodiments, the compound is (R)-2-(2-(methyl(4-(o-tolyl)quinolin-7-yl)amino)acetamido)propanamide, or a pharmaceutically acceptable salt thereof:

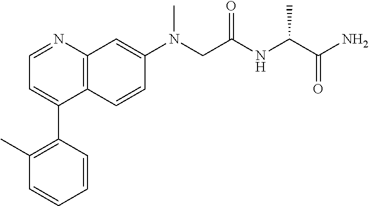 [SMILES: Cc1ccccc1-c1ccnc2cc(N(C)CC(=O)N[C@H](C)C(N)=O)ccc12]

In certain embodiments, the compounds inhibits POLRMT.

In certain embodiments, the compounds promote POLRMT.

The compounds of the present invention may contain asymmetric carbon atoms (sometimes as the result of a deuterium atom) and thereby can exist as either individual stereoisomers or mixtures of the enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and ketoenol tautomerism is possible, it is understood that the substituent might in fact be present, in whole or in part, in the oxo (=O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention. See, e.g., Gannes, L Z et al., Comp Biochem PhysiolMo/Integr Physiol, 119, 725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali et al, J. Med. Chem. 62, 5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto el al., J. Med Chem. 34, 2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 h,, preferably 10 min to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers. diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schutzenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated POLRMT modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds, a pharmaceutically acceptable salt of said compounds, or prodrugs of said compounds. In addition to the compound of the invention, a salt thereof, or a prodrug thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., Remington: The Science and Practice of Pharmacy, Elsevier Science, 23rd ed. (2020).

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

In another embodiment, a pharmaceutical composition of this invention further comprises a second therapeutic agent. The second therapeutic agent may be selected from any pharmaceutically active compound; preferably the second therapeutic agent is known to treat cancer, neurodegenerative disorders, or metabolic disorders. Alternatively, the compounds of the invention and second therapeutic agent may be administered together (within less than 24 hours of one another, consecutively or simultaneously) but in separate pharmaceutical compositions. In certain embodiments, the compounds on the invention and second therapeutic agent can be administered separately (e.g., more than 24 hours of one another). If the second therapeutic agent acts synergistically with the compounds of this invention, the therapeutically effective amount of such compounds and/or the second therapeutic agent may be less that such amount required when either is administered alone.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by mass spectrometry and/or NMR, where peaks assigned to the characteristic protons in the title compound are presented where appropriate. ¹H NMR shift (δ) are given in parts per million (ppm) down field from an internal reference standard.

The abbreviations used herein are known to a person of ordinary skill in the art. A partial list of abbreviations that may be used herein include: acetonitrile (MeCN), ammonium carbonate (NH₄)₂CO₃, ammonium chloride (NH₄Cl), aqueous (aq.), 1,1′-bis(diphenylphosphino)ferrocene (dppf), 1,3-bis(diphenylphosphino)propane (dppp), bis(pinacolato)diboron (B₂pin₂), N-bromosuccinimide (NBS), bromo-tris-pyrrolidino-phosphonium hexafluorophosphate (PyBroP), boron tribromide (BBr₃), butyl lithium (BuLi), Calcd. (Calcd.), cesium carbonate (Cs₂CO₃), dichloromethane (DCM, CH₂Cl₂), N,N-dicyclohexylcarbodiimide (DCC), dichloroethane (DCE), diethyl azodicarboxylate (DEAD), diisopropyl azodicarboxylate (DIAD), N,N-diisopropylethylamine (DIPEA), 4-dimethylaminopyridine (DMAP), dimethylformamide (DMF), dimethyl sulfoxide (DMSO), di-tert-butyl decarbonate (Boc₂O), 1-ethyl-3-(3-dimethylaminopropyl)carbodiimide (EDC), electrospray ionization (ESI), enantiomeric excess (ee), ethyl acetate (EtOAc), hour (h.), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), hydroxybenzotriazole (HOBt), isopropyl alcohol (IPA), lithium hydroxide monohydrate (LiOH·H₂O), methanol (MeOH), methyl iodide (Mel), minutes (min.), potassium carbonate (K₂CO₃), liquid chromatography-mass spectrometry (LCMS), phenyliodide(III) diacetate (PIDA), propylphosphonic anhydride (T₃P), reverse phase (RP), room/ambient temperature (rt, RT), silver oxide (Ag₂O), sodium hydride (NaH), sodium sulfate (Na2SO3), supercritical fluid chromatography (SFC), tetrahydrofuran (THF), triethylamine (Et₃N), thionyl chloride (SOCl₂), triphenylphosphine (PPh), dicyclohexyl[2′,4′,6′-tris(propan-2-yl)[1,1′-biphenyl]-2-yl]phosphane (XPhos).

Table 1 provides a listing of exemplary compounds of the present invention and their ICso values for inhibition of POLRMT.

Example 1-2: methyl 4-(o-tolyl)quinoline-7-carboxylate and 4-(o-tolyl)quinoline-7-carboxamide

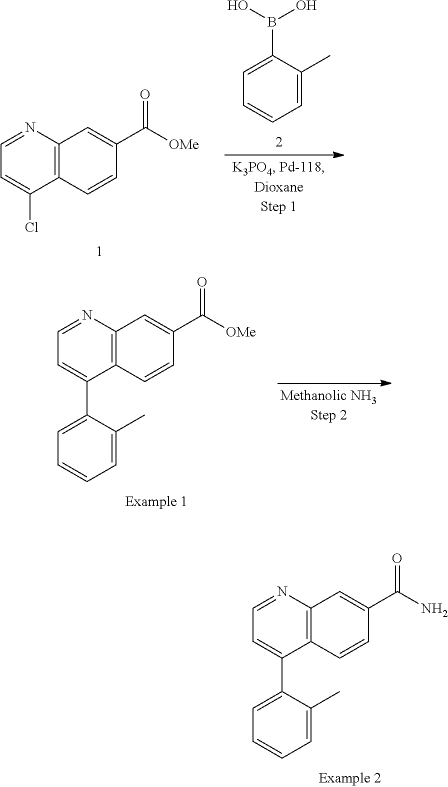

Synthesis of methyl 4-(o-tolyl)quinoline-7-carboxylate, Example 1 [Step 1]: A stirred solution of methyl 4-chloroquinoline-7-carboxylate (1, 400 mg, 2 mmol) in 1,4-dioxane (10 mL) was added 2-tolylboronic acid (2, 500 mg, 4 mmol) and K₃PO₄ (1.2 g, 5.5 mmol) was purged with argon for 15 min. Pd-118 (120 mg, 0.2 mmol) was then added and the mixture was stirred for 12 h at 80° C. It was then cooled to ambient temperature, diluted with water and extracted with EtOAc. The organic layer was separated, washed with brine, dried over anhydrous sodium sulfate, filtered, and concentrated in vacuo. The product was purified by flash column chromatography on silica gel (eluent: 10% EtOAc in hexanes) to afford methyl 4-(o-tolyl)quinoline-7-carboxylate (Example 1, 600 mg). LCMS (ESI): calculated for C₁₈H₁₅NO₂: 277; found: [M+H]⁺: 278. ¹H NMR (400 MHz, DMSO-d₆) δ 9.08 (d, 1H), 8.67 (s, 1H), 8.03 (dd, 1H), 7.54-7.52 (m, 2H), 7.46-7.43 (m, 2H), 7.40-7.36 (m, 1H), 7.26 (d, 1H), 3.94 (s, 3H), 1.98 (s, 3H).

Synthesis of 4-(o-tolyl)quinoline-7-carboxamide, Example 2 [Step 2]: Neat methanolic NH₃ (7M) (0.5 mL, 3.3 mmol) was added to a stirred solution of methyl 4-(o-tolyl)quinoline-7-carboxylate (Example 1, 90 mg, 0.3 mmol) and the reaction mixture heated to 60° C. for 18 h. After completion of the reaction, volatiles are removed under reduced pressure. The product was purified by flash column chromatography on silica gel (20% EtOAc in hexanes as eluent) to give 4-(o-tolyl)quinoline-7-carboxamide (Example 2, 50 mg). LCMS (ESI): calculated for C₁₇H₁₄N₂O: 235; found: [M+H]⁺: 236. ¹H NMR (400 MHz, DMSO-d₆) δ 9.03 (d, 1H), 8.65 (s, 1H), 8.30 (br s, 1H), 7.98 (d, 1H), 7.60 (br s, 1H), 7.47-7.34 (m, 5H), 7.25 (d, 1H), 1.98 (s, 3H).

### Example 3-9: N-methyl-4-(o-tolyl)quinoline-7-carboxamide, N,N-dimethyl-4-(o-tolyl)quinoline-7-carboxamide, N-benzyl-N-methyl-4-(o-tolyl)quinoline-7-carboxamide, N-benzyl-4-(o-tolyl)quinoline-7-carboxamide, morpholino(4-(o-tolyl)quinolin-7-yl)methanone, (4-methylpiperazin-1-yl)(4-(o-tolyl)quinolin-7-yl)methanone, and 1-(4-(4-(o-tolyl)quinoline-7-carbonyl)piperazin-1-yl)ethanone

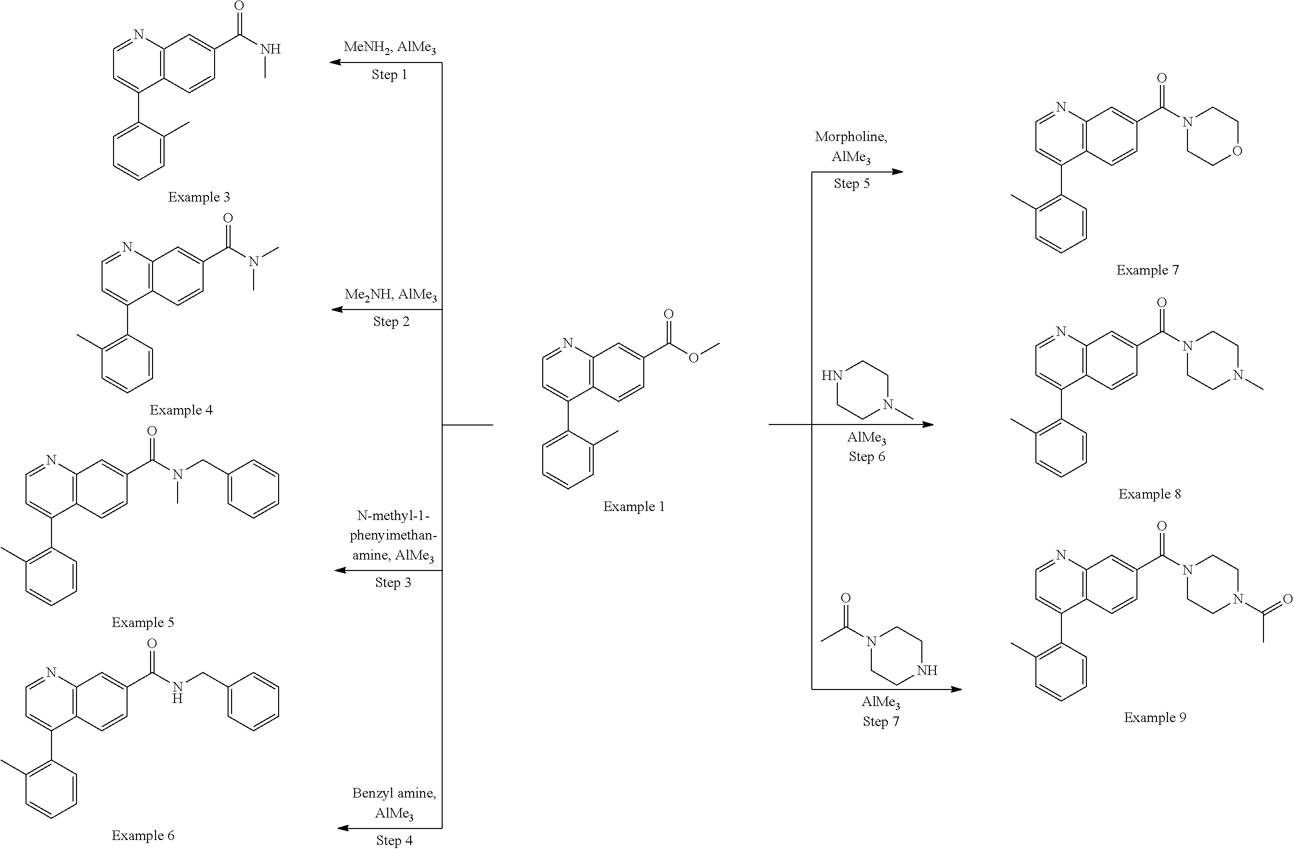

Synthesis of N-methyl-4-(o-tolyl)quinoline-7-carboxamide, Example 3 [step 1]: To a stirred solution of methyl 4-(o-tolyl)quinoline-7-carboxylate (Example 1, 100 mg, 0.4 mmol) and methylamine (2M in THF) (2 mL, 4 mmol) at 0° C. was added trimethyl aluminum (2M in toluene) (0.35 mL, 0.7 mmol). The reaction mixture was continued to stir for 16 h. and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield N-methyl-4-(o-tolyl)quinoline-7-carboxamide (Example 3, 34 mg) as a formate salt. LCMS(ESI) Calcd. for C₁₈H₁₆N₂O: 276; found: [M+H]⁺=277. ¹H NMR (400 MHz, DMSO-d₆) δ 9.03 (d, 1H), 8.77 (d, 1H), 8.58 (s, 1H), 8.52 (s, 1H), 7.95 (dd, 1H), 7.47-7.44 (m, 4H), 7.39-7.35 (m, 1H), 7.25 (d, 1H), 2.85 (d, 3H), 1.98 (s, 3H).

Synthesis of N,N-dimethyl-4-(o-tolyl)quinoline-7-carboxamide, Example 4 [step 2]: To a stirred solution of methyl 4-(o-tolyl)quinoline-7-carboxylate (Example 1, 150 mg, 0.5 mmol) and dimethylamine (2M in THF) (1.4 mL, 2.7 mmol) at 0° C. was added trimethyl aluminum (2M in toluene) (0.6 mL, 1.1 mmol). The reaction mixture was continued to stir for 16 h. and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield N,N-dimethyl-4-(0-tolyl)quinoline-7-carboxamide (Example 4, 80 mg). LCMS(ESI) Calcd. for C₁₉H₁₈N₂O: 296; found: [M+H]⁺=297. ¹H NMR (400 MHz, DMSO-d₆) δ 9.01 (d, 1H), 8.08 (s, 1H), 7.56 (d, 1H), 7.47-7.44 (m, 4H), 7.39-7.35 (m, 1H), 7.25 (d, 1H), 3.05 (s, 3H), 2.97 (s, 3H), 1.99 (s, 3H).

Synthesis of N-benzyl-N-methyl-4-(o-tolyl)quinoline-7-carboxamide, Example 5 [step 3]: To a stirred solution of methyl 4-(o-tolyl)quinoline-7-carboxylate (Example 1, 150 mg, 0.5 mmol) and N-methyl-1-phenyl-methanamine (0.35 mL, 2.7 mmol) at 0° C. was added trimethyl aluminium (2M in toluene) (0.6 mL, 1.1 mmol). The reaction mixture was continued to stir for 16 h. and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield N-benzyl-N-methyl-4-(o-tolyl)quinoline-7-carboxamide (Example 5, 80 mg). LCMS(ESI) Calcd. for C₂₅H₂₂N₂O: 366; found: [M+H]⁺=367. ¹H NMR (400 MHz, DMSO-d₆) (at 100° C.) δ 9.00 (d, 1H), 8.12 (s, 1H), 7.58 (d, 1H), 7.48-7.23 (m, 1H), 4.67 (s, 2H), 2.95 (s, 3H), 2.01 (s, 3H).

Synthesis of N-benzyl-4-(o-tolyl)quinoline-7-carboxamide, Example 6 [step 4]: To a stirred solution of methyl 4-(o-tolyl)quinoline-7-carboxylate (Example 1, 150 mg, 0.5 mmol) and benzylamine (0.30 mL, 2.7 mmol) at 0° C. was added trimethyl aluminium (2M in toluene) (0.6 mL, 1.1 mmol). The reaction mixture was continued to stir for 16 h. and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield N-benzyl-4-(o-tolyl)quinoline-7-carboxamide (Example 6, 52 mg). LCMS(ESI) Calcd. for C₂₄H₂₀N₂O: 352; found: [M+H]⁺=353. ¹H NMR (400 MHz, DMSO-d₆) δ 9.39 (t, 1H), 9.04 (d, 1H), 8.67 (d, 1H), 8.0 (dd, 1H), 7.48-7.42 (m, 4H), 7.39-7.34 (m, 5H), 7.25 (d, 2H), 4.54 (d, 2H), 1.98 (s, 3H).

Synthesis of morpholino-[4-(o-tolyl)-7-quinolyl]methanone, Example 7 [step 5]: To a stirred solution of methyl 4-(o-tolyl)quinoline-7-carboxylate (Example 1, 150 mg, 0.5 mmol) and morpholine (0.3 mL, 3.0 mmol) at 0° C. was added trimethyl aluminium (2M in toluene) (0.6 mL, 1.1 mmol). The reaction mixture was continued to stir for 16 h. and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield morpholino-[4-(o-tolyl)-7-quinolyl]methanone (Example 7, 78 mg). LCMS(ESI) Calcd. for C₂₁H₂₀N₂O₂: 332; found: [M+H]⁺=333. ¹H NMR (400 MHz, DMSO-d₆) (at 100° C.) δ 9.02 (d, 1H), 8.10 (s, 1H), 7.55-7.37 (m, 6H), 7.24 (d, 1H), 3.64 (d, 4H), 3.56 (s, 4H), 2.03 (s, 3H).

Synthesis of (4-methylpiperazin-1-yl)-[4-(o-tolyl)-7-quinolyl]methanone, Example 8 [step 6]: To a stirred solution of methyl 4-(o-tolyl)quinoline-7-carboxylate (Example 1, 150 mg, 0.5 mmol) and 1-methylpiperazine (0.30 mL, 3 mmol) at 0° C. was added trimethyl aluminum (2M in toluene) (0.6 mL, 1.1 mmol). The reaction mixture was continued to stir for 16 h. and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield (4-methylpiperazin-1-yl)-[4-(o-tolyl)-7-quinolyl]methanone (Example 8, 75 mg). LCMS(ESI) Calcd. for C₂₂H₂₃N₃O: 345; found: [M+H]⁺=346. ¹H NMR (400 MHz, DMSO-d₆) (at 100° C.) δ 9.00 (d, 1H), 8.06 (s, 1H), 7.53-7.35 (m, 6H), 7.24 (d, 1H), 3.55 (s, 4H), 2.38 (t, 4H), 2.24 (s, 3H), 2.03 (s, 3H).

Synthesis of 1-(4-(4-(o-tolyl)quinoline-7-carbonyl)piperazin-1-yl)ethan-1-one, Example 9 [step 7]: To a stirred solution of methyl 4-(o-tolyl)quinoline-7-carboxylate (Example 1, 150 mg, 0.5 mmol) and 1-piperazin-1-ylethanone (0.3 mL, 3 mmol) at 0° C. was added trimethyl aluminium (2M in toluene) (0.6 mL, 1.1 mmol). The reaction mixture was continued to stir for 16 h, and then quenched with aqueous NH₄Cl solution and extracted with EtOAc. Organic layer was separated and further washed with brine, dried over anhydrous sodium sulfate, filtered, and concentrated under reduced pressure. The product was purified by reverse phase prep-HPLC and lyophilized to yield 1-(4-(4-(o-tolyl)quinoline-7-carbonyl)piperazin-1-yl)ethan-1-one (Example 9, 75 mg, 37%) as an off white solid. LCMS(ESI) Calcd. for C₂₃H₂₃N₃O₂: 373; found: [M+H]⁺=374. ¹H NMR (400 MHz, DMSO-d₆) (at 100° C.) δ 9.01 (d, 1H), 8.12 (s, 1H), 7.56-7.37 (m, 6H), 7.24 (d, 1H), 3.55 (s, 8H), 2.02 (s, 6H).

### Example 10

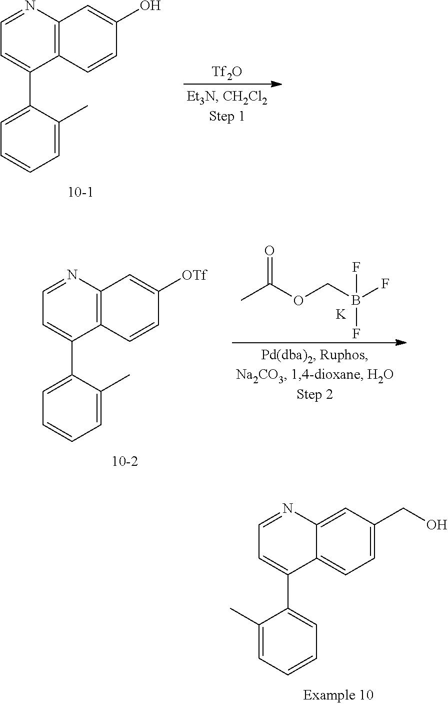

Synthesis of 4-(o-tolyl)quinolin-7-yl trifluoromethanesulfonate [Step 1]: To a stirred solution of 4-(o-tolyl)quinolin-7-ol (250 mg, 1.06 mmol) in DCM (15 mL) was added (0.19 mL, 2.34 mmol) followed by trifluoromethanesulfonic anhydride (0.39 mL, 2.34 mmol) at 0° C. and stirred at RT for 2 h. Reaction mixture was quenched with a saturated aqueous ammonium chloride solution. Organic layer was dried over sodium sulphate and evaporated. The crude was purified by flash column chromatography to get [4-(o-tolyl)-7-quinolyl]trifluoromethanesulfonate (300 mg). LCMS (ESI) Calcd. for C₁₇H₁₂F₃NO₃S: 367, found[M+H]⁺=368; ¹H NMR (400 MHz, DMSO-d₆): δ 9.10-9.09 (s, 1H), 8.24-8.23 (s, 1H), 7.71-7.69 (m, 1H), 7.60-7.54 (m, 2H), 7.46-7.45 (m, 2H), 7.40-7.38 (m, 1H), 7.28-7.26 (m, 1H), 1.98 (s, 3H).

Synthesis of (4-(o-tolyl)quinolin-7-yl)methanol [Step 2]: To a stirred solution of [4-(o-tolyl)-7-quinolyl]trifluoromethanesulfonate (200 mg, 0 5 mmol) and potassium (acetoxymethyl)trifluoroborate (98 mg, 0.5 mmol) in 1,4-Dioxane (6 mL) and water (1 mL) was added Na₂CO₃ (173 mg, 1.63 mmol) and degassed with argon for 5 min. Ruphos (51 mg, 0 1 mmol) followed by Pd(dba)₂ (31 mg, 0.05 mmol) was added and stirred at 100° C. for 12 h. Reaction mixture was cooled and diluted with EtOAc and washed with water and brine, dried over anhydrous sodium sulfate and concentrated in vacuo. The crude was purified by prep HPLC purification and lyophilized to afford (4-(o-tolyl)quinolin-7-yl)methanol (30 mg). LCMS (ESI) Calcd. for C₁₇H₁₅NO: 249, found [M+H]⁺=250; ¹H NMR (400 MHz, DMSO-d₆): δ 8.93-8.92 (d, 1H), 8.02 (s, 1H), 7.50-7.48 (d, 1H), 7.45-7.40 (m, 2H), 7.37-7.33 (m, 3H), 7.23-7.21 (d, LH), 5.44 (br s, 1H), 4.7 (s, 2H), 1.97 (s, 3H).

### Example 11

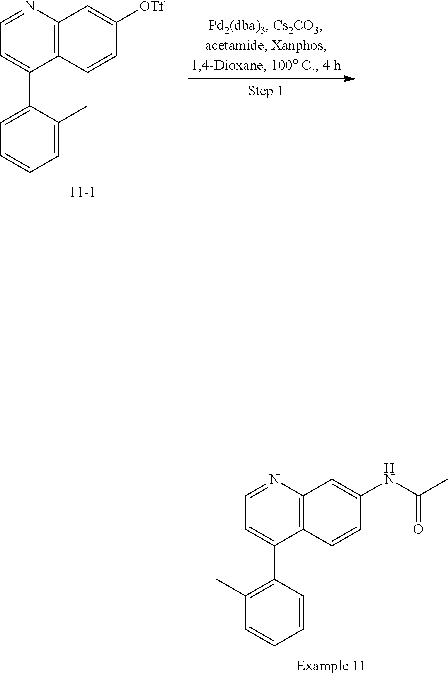 [SMILES: Cc1ccccc1-c1ccnc2cc(OS(=O)(=O)C(F)(F)F)ccc12 ; CC(=O)Nc1ccc2c(-c3ccccc3C)ccnc2c1]

Synthesis of N-(4-(o-tolyl)quinolin-7-yl)acetamide [step 1]: To a suspension of 4-(o-tolyl)quinolin-7-yl trifluoromethanesulfonate (150 mg, 0.4 mmol) in dioxane (4 mL) was added cesium carbonate (265 mg, 0.8 mmol), acetamide (30 mg, 0.5 mmol) and Xanphos (12 mg, 0.02 mmol) and degassed with nitrogen for 10 min. Then Pd₂(dba)₃ (20 mg, 0.02 mmol) was added and the reaction mixture was heated 100° C. for 4 h. The reaction mixture was cooled to room temperature and filtered through celite bed. It was washed with ethyl acetate (2×15 mL). The organic layer was evaporated under reduced pressure. The crude obtained was purified by RP prep-HPLC purification and lyophilized to get N-(4-(o-tolyl)quinolin-7-yl)acetamide (35 mg). LCMS(ESI) Calcd. for C₁₈H₁₆N₂O: 276, found [M+H]⁺: 277; ¹H NMR (400 MHz, DMSO-d₆): δ 10.31 (s, 1H), 8.67 (d, 1H), 8.50 (d, 1H), 7.61-7.58 (m, 1H), 7.42-7.41 (m, 2H), 7.37-7.30 (m, 2H), 7.24-7.21 (m, 2H), 2.11 (s, 3H), 1.97 (s, 3H).

### Example 12-15

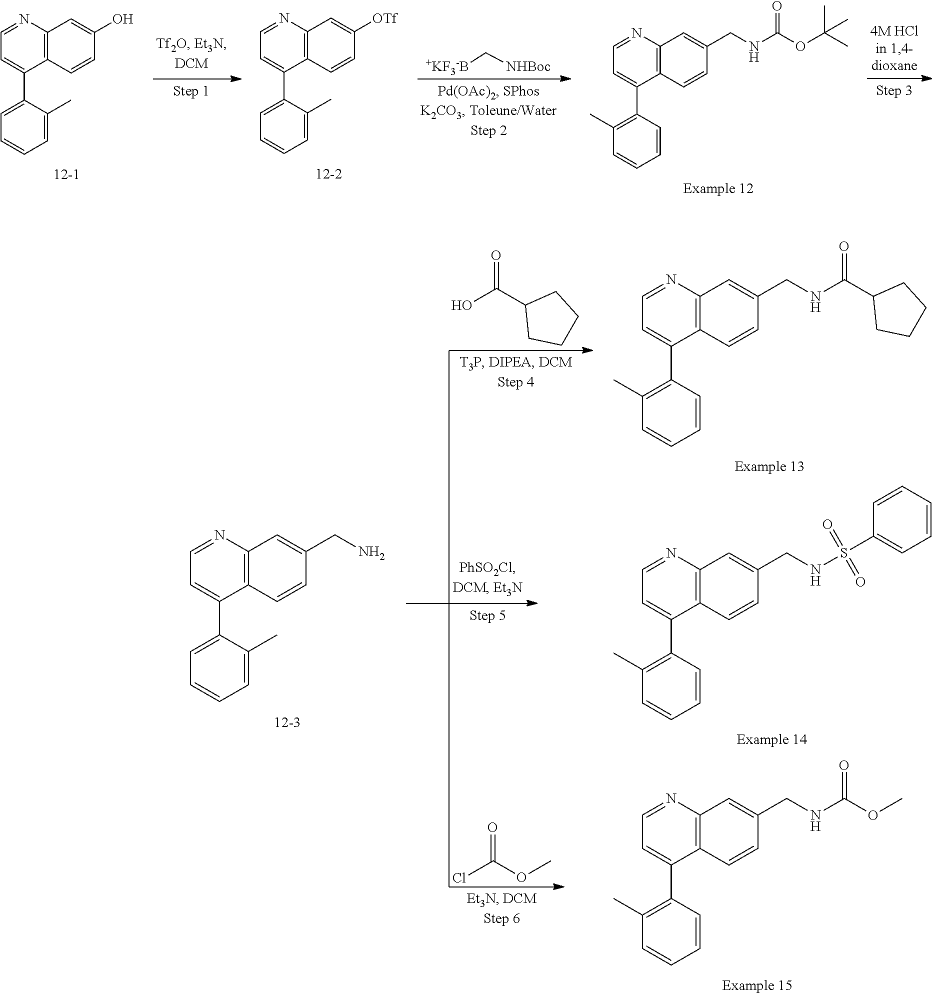

Synthesis of 4-(o-tolyl)quinolin-7-yl trifluoromethanesulfonate [Step 1]: To a stirred solution of 4-(o-tolyl)quinolin-7-ol (1.50 g, 6.4 mmol) in DCM (15 mL) was added pyridine (1.1 mL, 14.0 mmol) followed by trifluoromethanesulfonic anhydride (2.4 mL, 14.0 mmol) at 0° C. and stirred at RT for 1 h. Volatiles were removed under reduced pressure. The crude was partitioned between DCM and water. Organic layer was washed with water and brine. It was dried over magnesium sulphate and concentrated under reduced pressure. The crude was purified via flash column chromatography to afford 4-(o-tolyl)quinolin-7-yl trifluoromethanesulfonate (1.90 g). LCMS: calculated for C₁₇H₁₂F₃NO₃S: 367, found [M+H]⁺: 368; ¹H NMR (400 MHz, DMSO-d₆) δ 9.09 (d, 1H), 8.23 (d, 1H), 7.71 (dd, 1H), 7.59 (d, 1H), 7.55 (d, 1H), 7.44 (t, 2H), 7.39 (t, 1H), 7.28 (d, 1H), 1.98 (s, 3H).

Synthesis of tert-butyl ((4-(o-tolyl)quinolin-7-yl)methyl)carbamate [Step 2]: To a solution of 4-(o-tolyl)quinolin-7-yl trifluoromethanesulfonate (250 mg, 0.7 mmol) in toluene (3 mL) and water (0.7 mL) mixture was added K₂CO₃ (280 mg, 2.0 mmol) and degassed with nitrogen for 5 min. Then (tert-butoxycarbonylamino)methyl-trifluoro-potassio-boron (240 mg, 1.0 mmol) followed by SPhos (30 mg, 0.07 mmol) and Pd(OAc)₂ (8.0 mg, 0.03 mmol) were added and heated at 110 C for 16 h. The reaction mixture was filtered and washed with EtOAc. The organic layer was concentrated under reduced pressure. The crude obtained was purified via RP prep HPLC purification to afford tert-butyl ((4-(o-tolyl)quinolin-7-yl)methyl)carbamate (26 mg). ¹H NMR (400 MHz, DMSO-d₆) δ 8.93 (d, 1H), 7.90 (s, 1H), 7.59 (t, 1H), 7.45-7.40 (m, 3H), 7.38-7.33 (m, 3H), 7.22 (d, 1H), 4.33 (d, 2H), 1.97 (s, 3H), 1.40 (s, 9H). LC/MS: calculated for C₂₂H₂₄N₂O₂: 348, found [M+H]⁺: 349.

Synthesis of (4-(o-tolyl)quinolin-7-yl)methanamine [Step 3]: To a solution of tert-butyl ((4-(o-tolyl)quinolin-7-yl)methyl)carbamate (30 mg, 0.08 mmol) in 1,4-dioxane (2 mL) was added 4M HCl in 1,4-dioxane (0.2 mL) dropwise at 0° C. and stirred at RT for 4 h. The crude reaction was dried in vacuum to get crude (4-(o-tolyl)quinolin-7-yl)methanamine (15 mg). LCMS: calculated for C₁₇H₁₆N₂: 248, found [M+H]⁺: 249; ¹H NMR (400 MHz, DMSO-d₆) δ 9.11 (d, 1H) 8.57 (br s, 2H), 8.33 (s, 1H), 7.74 (d, 1H), 7.61 (d, 1H), 7.52-7.45 (m, 3H), 7.40 (t, 1H), 7.25 (d, 1H), 4.30 (s, 2H), 1.99 (s, 3H).

N-((4-(o-tolyl)quinolin-7-yl)methyl)cyclopentanecarboxamide [Step 4]: A round bottom flask was charged with (4-(o-tolyl)quinolin-7-yl)methanamine (60 mg, 0.2 mmol), cyclopentanecarboxylic acid (30 mg, 0.2 mmol), and DIPEA (0.07 mL, 0.5 mmol) in DCM (1.5 mL). The solution was cooled to 0° C. A 50% solution of T₃P (0.28 mL, 0.48 mmol) in ethyl acetate was added and stirred at RT for 3 h. The reaction mixture was diluted with DCM and washed with water and brine, dried over Na2SO4 and concentrated under reduced pressure. The crude was purified via RP prep HPLC purification to afford N-((4-(o-tolyl)quinolin-7-yl)methyl)cyclopentanecarboxamide (10 mg). LCMS: calculated for C₂₃H₂₄N₂O: 344, found [M+H]⁺: 345: ¹H NMR (400 MHz, DMSO-d₆) δ 8.93 (d, 1H), 8.47 (t, 1H), 7.90 (s, 1H), 7.43-7.41 (m, 3H), 7.38-7.33 (m, 3H) 7.21 (d, 1H), 4.47 (d, 2H), 2.68-2.64 (m, 1H), 1.97 (s, 3H), 1.83-1.77 (m, 2H), 1.70-1.62 (m, 4H), 1.56-1.53 (m, 2H).

Synthesis of N-((4-(o-tolyl)quinolin-7-yl)methyl)benzenesulfonamide [Step 5]: A round bottom flask was charged with (4-(o-tolyl)quinolin-7-yl)methanamine (100 mg, 0.4 mmol) in DCM (2 mL) at 0° C. followed by Et₃N (0.11 mL, 0.8 mmol) and benzenesulfonyl chloride (0.08 mL, 0.6 mmol) were added dropwise. The reaction mixture was warmed up to RT and stirred for 2 h. The reaction mixture was diluted with DCM and washed with water and brine, dried over Na2SO₄ and concentrated under reduced pressure. The crude was purified via RP prep HPLC purification to afford N-((4-(o-tolyl)quinolin-7-yl)methyl)benzenesulfonamide (16 mg). LCMS: calculated for C₂₃H₂₀N₂O₂S: 388, found [M+H]⁺: 389; ¹H NMR (400 MHz, DMSO-d₆) δ 8.92 (d, 1H), 8.32 (br s, 1H), 7.95 (s, 1H), 7.79 (d, 2H), 7.56-7.50 (m, 3H), 7.43-7.33 (m, 5H), 7.26 (d, 1H), 7.20 (d, 1H), 4.20 (s, 2H), 1.96 (s, 3H).

Synthesis of methyl ((4-(o-tolyl)quinolin-7-yl)methyl)carbamate [Step 6] To a stirred solution of (4-(o-tolyl)quinolin-7-yl)methanamine (40 mg, 0.2 mmol) in DCM (2 mL) was added Et3N (0.05 mL, 0.32 mmol) followed by methyl carbonochloridate (0.02 mL, 0.2 mmol) at 0° C. and stirred at RT for 2 h. The reaction mixture was diluted with DCM and washed with brine. It was dried over Na₂SO₄ and concentrated under reduced pressure and purified by RP prep HPLC purification to afford methyl ((4-(o-tolyl)quinolin-7-yl)methyl)carbamate (7 mg). LC/MS: calculated for C₁₉H₁₈N₂O₂: 306, found [M+H]⁺: 307; ¹H NMR (400 MHz, DMSO-d₆) δ 8.93 (d, 1H), 7.93 (s, 1H), 7.86 (s, 1H), 7.46-7.42 (m, 3H), 7.37-7.34 (m, 3H), 7.21 (d, 1H), 4.39 (d, 2H), 3.57 (s, 3H), 1.97 (s, 3H).

### Example 16

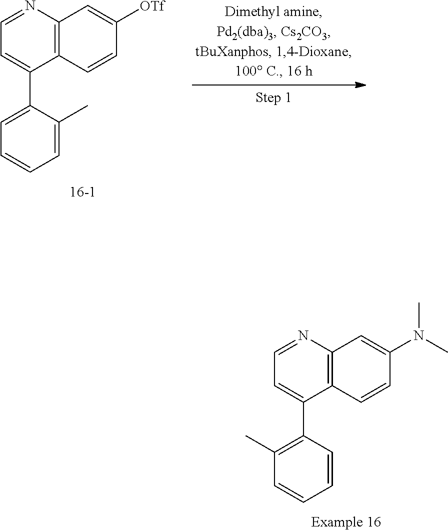

Synthesis of N,N-dimethyl-4-(o-tolyl)quinolin-7-amine [Step 1]: To a suspension of 4-(o-tolyl)quinolin-7-yl trifluoromethanesulfonate (100 mg, 0.3 mmol) in dioxane (4 mL) was added cesium carbonate (265 mg, 0.8 mmol), dimethylamine (2 M in THF) (1.4 mL, 2.7 mmol) and t-BuXanphos (45 mg, 0.1 mmol) and degassed with nitrogen for 10 min. Then Pd₂(dba)₃ (25 mg, 0.03 mmol) was added and the reaction mixture was heated 100° C. for 4 h. The reaction mixture was cooled to room temperature and filtered through celite bed. The bed was washed with ethyl acetate (2×15 mL). The organic layer was evaporated under reduced pressure. The crude obtained was purified by RP prep-HPLC purification to get N,N-dimethyl-4-(o-tolyl)quinolin-7-amine (12 mg). LCMS(ESI) Calcd. for C₁₈H₁₈N₂:262, found [M+H]=263; ¹H NMR (400 MHz, DMSO-d₆): δ 8.71 (d, 1H), 7.40-7.39 (m, 2H), 7.35-7.31 (m, 1H), 7.22-7.15 (m, 3H), 7.07-7.06 (m, 1H), 6.98 (d, 1H), 3.04 (s, 6H), 1.98 (s, 3H).

### Example 17-18

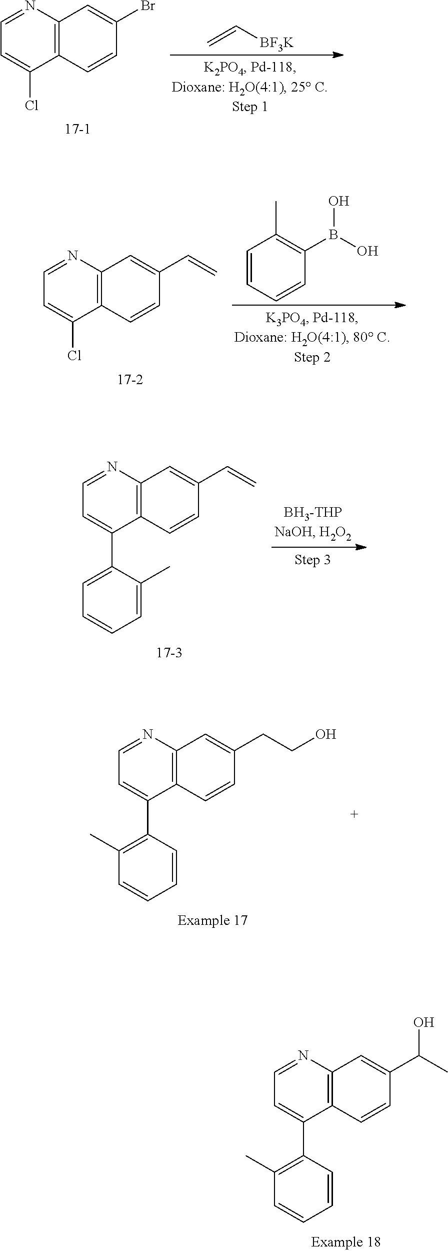

Synthesis of 4-chloro-7-vinylquinoline [Step 1]: To a solution of 7-bromo-4-chloro-quinoline (5.0 g, 20.6 mmol) and potassium vinyltrifluoroborate (4.1 g, 30.9 mmol) in 1,4-dioxane (120 mL) was added a solution of K₃PO₄ (10.9 g, 51.5 mmol) in water (30 mL) at 0° C. It was degassed with nitrogen for 15 min. Then PdCl₂(dtbpf) (Pd-118, 1.3 g, 2.0 mmol) was added to it. The reaction mass was stirred at room temperature for 3 h. LCMS of crude reaction mass confirmed the formation of desired product. Solvent was evaporated under reduced pressure. It was partitioned between EtOAc and water. Organic layer was collected. Aqueous layer was further extracted with EtOAc (twice). Combined organic layer was washed with brine, dried over Na₂SO₄ and concentrated under reduced pressure. The crude was purified by flash column chromatography using 20% ethyl acetate in hexanes to get 4-chloro-7-vinylquinoline (3.0 g). LCMS (ESI) Calcd. for C₁₁H₈ClN: 189, found [M+H]⁺=190; ¹H NMR (400 MHz, DMSO-d₆) δ 8.81 (d, 1H), 8.14 (d, 1H), 8.07 (s, 1H), 7.97 (d, 1H), 7.77-7.65 (m, 1H), 7.00 (dd, 1H), 6.13 (d, 1H), 5.51 (d, 1H)

Synthesis of 4-(o-tolyl)-7-vinylquinoline [Step 2]: To a solution of 4-chloro-7-vinylquinoline (230 mg, 1.2 mmol) and o-tolylboronic acid (250 mg, 1.8 mmol) in 1,4-dioxane (6 mL) was added a solution of KiPO₄ (645 mg, 3.0 mmol) in water (1.5 mL) at 0° C. It was degassed with nitrogen for 10 min. Then PdCl₂(dtbpf) (80 mg, 0.12 mmol) was added to it. The reaction mass was stirred at 80° C. temperature for 2 h. LCMS of crude reaction mass confirmed the formation of desired product. Solvent was evaporated under reduced pressure. It was partitioned between EtOAc and water. Organic layer was collected. Aqueous layer was further extracted with EtOAc (twice). Combined organic layer was washed with brine, dried over Na2SO4 and concentrated under reduced pressure. The crude was purified by flash column chromatography using 30% ethyl acetate in hexanes to get 4-(o-tolyl)-7-vinyl-quinoline (190 mg). LCMS (ESI) Calcd. for C₁₈H₁₅N: 245, found [M+H]⁺=246; ¹H NMR (400 MHz, DMSO-d₆) δ 9.01 (s, 1H), 8.10 (s, 1H), 7.83 (d, 1H), 7.53-7.32 (m, 5H), 7.25 (d, 1H), 7.09-6.90 (m, 1H), 6.08 (d, 1H), 5.50 (d, 1H), 1.99 (s, 3H).

Synthesis of 2-(4-(o-tolyl)quinolin-7-yl)ethan-1-ol [Step 3]: To a stirred solution of 4-(o-tolyl)-7-vinyl-quinoline (300 mg, 1.2 mmol) in THF (3 mL), borane-tetrahydrofuran complex solution 1.0 M in THF (3.6 mL, 3.6 mmol) was added at 0° C. Then reaction mixture was stirred at room temperature for 16 h. After 16 h, 3M NaOH solution (1.2 mL, 3.67 mmol) and H₂O₂ (1.2 mL) were added to the reaction mixture at 0° C. Then reaction mixture was allowed to come to room temperature and then heated at 80° C. for 2 h. After that it was washed with water and extracted with ethyl acetate. The organic phase was dried over Na2SO4, filtered and evaporated to yield crude residue. The crude compound was purified by combi-column chromatography using 0-50/o EA/hexanes as eluent to get 2-(4-(o-tolyl)quinolin-7-yl)ethan-1-ol (100 mg, 31%) as a solid. LCMS (ESI) Calcd. for C₁₈H₁₇NO: 263, found [M+H]⁺=264; ¹H NMR (400 MHz, DMSO-d₆) δ 8.90 (d, 1H), 7.93 (s, 1H), 7.43-7.41 (m, 3H), 7.36-7.26 (m, 3H), 7.21 (d, 1H), 4.71 (t, 1H), 3.72 (q, 2H), 2.93 (t, 2H), 1.98 (s, 3H).

Another product was isolated from this reaction mixture which was 1-(4-(o-tolyl)quinolin-7-yl)ethan-1-ol (90 mg). LCMS (ESI) Calcd. for CIRH7NO: 263, found [M+H]⁺=264; ¹H NMR (400 MHz, DMSO-d₆) δ 8.93 (d, 1H), 8.02 (d, 1H), 7.54 (t, 1H), 7.45-7.41 (m, 2H), 7.37-7.35 (m, 3H), 7.32 (d, 1H), 5.38 (d, 1H), 4.94-4.90 (m, 1H),, 1.97 (s, 3H), 1.42-1.23 (m, 3H).

### Example 19

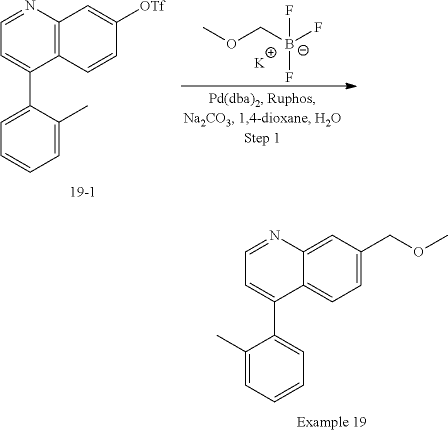

Synthesis of 7-(methoxymethyl)-4-(o-tolyl)quinoline [Step 1]: To a stirred solution of [4-(o-tolyl)-7-quinolyl]trifluoromethanesulfonate (200 mg, 0.5 mmol) and potassium methoxymethyltrifluoroborate (85 mg, 0.5 mmol) in 1,4-dioxane (6 mL) and water (1 mL) mixture, Na₂CO₃ (175 mg, 1.6 mmol) was added and degassed with argon for 5 min. Ruphos (50 mg, 0.1 mmol) followed by Pd(dba)₂ (30 mg, 0.05 mmol) was added and stirred at 100° C. for 12 h. Reaction mixture was cooled and diluted with EtOAc and washed with water and brine, dried over anhydrous sodium sulfate and concentrated under vacuo. The crude was purified by RP prep HPLC purification and lyophilized to afford 7-(methoxymethyl)-4-(o-tolyl)quinoline (14 mg). LCMS (ESI) Calcd. for C₁₈H₁₇NO. 263, found [M+H]⁺=264; ¹H NMR (400 MHz, DMSO-d₆): δ 8.95-8.94 (s, 1H), 8.01 (s, 1H), 7.51-7.50 (d, 1H), 7.44-7.41 (m, 2H), 7.38-7.34 (m, 3H), 7.24-7.22 (d, 1H), 4.63 (s, 2H), 3.36 (s, 3H), 1.97 (s, 3H).

### Example 20

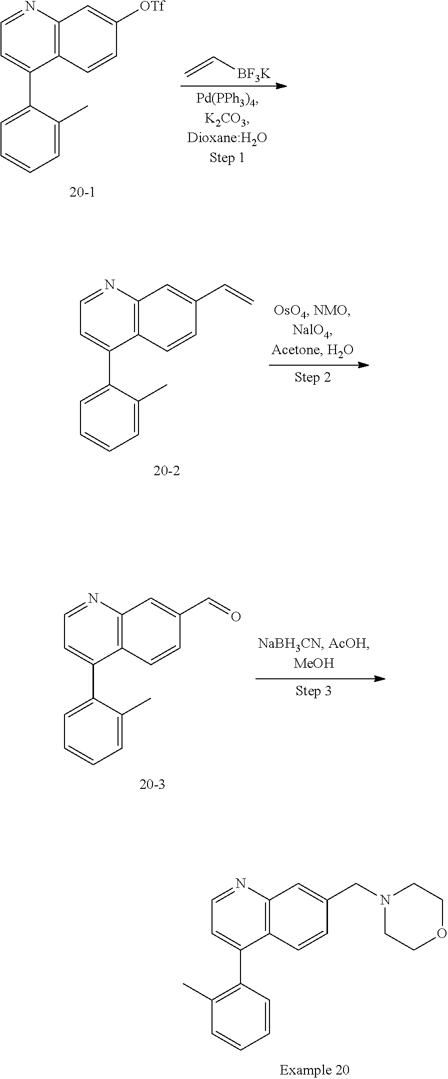

Synthesis of 4-(o-tolyl)-7-vinylquinoline [Step 1]: To a degassed solution of 4-(o-tolyl)quinolin-7-yl trifluoromethanesulfonate (3.0 g, 8.2 mmol) in 1,4-dioxane (12 mL) & water (3 mL) mixture was added K₂CO₃ (3.4 g, 24.5 mmol) followed by potassium vinyl trifluroborate (1.4 g, 10.6 mmol) at RT & degassed for 10 min with argon gas. Then Pd(PPh₃)₄(945 mg, 0.8 mmol) was added and heated at 110° C. for 12 h. The reaction mixture was filtered through celite bed and washed with EtOAc. The filtrate was washed with water, dried over anhydrous sodium sulfate and concentrated in vacuum. The crude was purified in combiflash chromatography to get 4-(o-tolyl)-7-vinyl-quinoline (1.2 g). LCMS (ESI) Calcd. for C₁₈H₁₅N: 245, found [M+H]⁺=246, ¹H NMR (400 MHz, DMSO-d₆): δ 8.94-8.93 (d, 1H), 8.07 (s, 1H), 7.77-7.76 (d, 1H), 7.44-7.41 (m, 2H), 7.36-7.34 (m, 3H), 7.24-7.23 (d, 1H), 7.02-6.95 (m, 1H), 6.07-6.02 (d, 1H), 5.47-5.44 (d, 1H), 1.98 (s, 3H).

Synthesis of 4-(o-tolyl)quinoline-7-carbaldehyde [Step 2]: To a solution of 4-(o-tolyl)-7-vinyl-quinoline (1.2 g, 4.9 mmol) in acetone (18 mL) and water (2 mL) mixture, was added NMO (1.2 g, 9.8 mmol) followed by OsO₄, 25% in H₂O (0.12 mg, 0.5 mmol) and stirred at 0° C. for 2 h. NaIO₄ (5.2 g, 24.5 mmol) was added to the reaction mixture at 0° C. and stirred at RT for 2 h. The reaction mixture was filtered through celite bed and washed with EtOAc. The organic layer was washed with water, dried over Na2SO₄ and concentrated to get 4-(o-tolyl)quinoline-7-carbaldehyde (850 mg). LCMS (ESI) Calcd. for C₁₇H₁₃NO: 247, found [M+H]⁺=248; ¹H NMR (400 MHz, DMSO-d₆): δ 10.27 (s, 1H), 9.12-9.11 (d, 1H), 8.72 (s, 1H), 7.95-7.93 (d, 1H), 7.57-7.53 (m, 2H), 7.46-7.43 (m, 2H), 7.40-7.37 (m, 1H), 7.27-7.25 (d, 1H), 1.98 (s, 3H).

Synthesis of 4-((4-(o-tolyl)quinolin-7-yl)methyl)morpholine [Step 3]: To a solution of 4-(o-tolyl)quinoline-7-carbaldehyde (150 mg, 0.6 mmol) in methanol (10 mL) was added morpholine (0.05 mL, 0.6 mmol) followed by AcOH (0.05 mL) at RT and stirred for 16 h. Sodium cyanoborohydride (55 mg, 0.9 mmol) was added and stirred at RT for 4 h. The reaction mixture was concentrated under reduced pressure and diluted with EtOAc and washed with water and brine, dried over anhydrous sodium sulfate and concentrated in vacuum. The crude was purified by RP prep HPLC purification and lyophilized to afford 4-((4-(o-tolyl)quinolin-7-yl)methyl)morpholine (8.0 mg). LCMS (ESI) Calcd. for C₂₁H₂₂N₂O: 318, found [M+H]⁺=319; ¹H NMR (400 MHz, DMSO-d₆): δ 8.92 (br s, 1H), 7.99 (s, 1H), 7.54-7.53 (m, 1H), 7.43-7.41 (m, 2H), 7.36-7.34 (m, 3H), 7.23-7.21 (m, 1H), 3.68-3.67 (m, 2H), 3.59-3.57 (m, 4H), 2.42-2.41 (m, 4H), 1.97 (s, 3H).

### Example 21

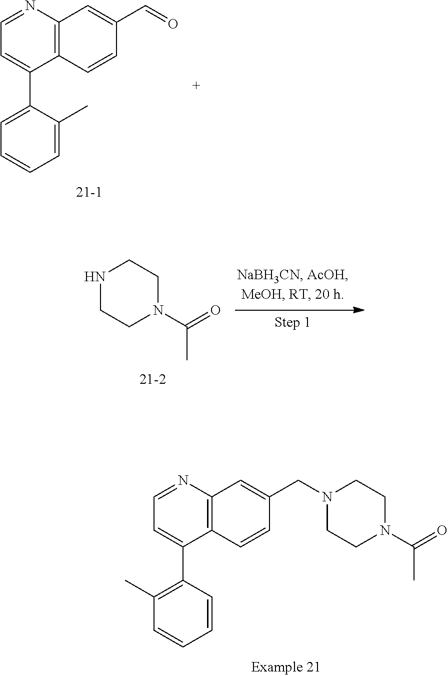

Synthesis of 1-(4-((4-(o-tolyl)quinolin-7-yl)methyl)piperazin-1-yl)ethan-1-one [Step 1]: To a solution of 4-(o-tolyl)quinoline-7-carbaldehyde (150 mg, 0.6 mmol) in methanol (2 mL) was added 1-piperazin-I-ylethanone (80 mg, 0.6 mmol) followed by AcOH (0.05 mL) at RT and stirred for 16 h. Sodium cyanoborohydride (60 mg, 1.0 mmol) was added to it and stirred at RT for 4 h. The reaction mixture was concentrated under reduced pressure. It was partitioned between EtOAc and water. Organic layer was collected and washed with brine, dried over anhydrous sodium sulfate and concentrated. The crude was purified via RP prep HPLC purification and lyophilized to afford 1-(4-((4-(o-tolyl)quinolin-7-yl)methyl)piperazin-1-yl)ethan-1-one (20 mg). LCMS (ESI) Calcd. for C₂₃H₂₅N₃O: 359, found [M+H]⁺=360; ¹H NMR (400 MHz, DMSO-d₆): δ 8.94-8.92 (s, 1H), 7.99 (s, I H), 7.54 (d, 1H), 7.43-7.42 (m, 2H), 7.37-7.33 (m, 3H), 7.23-7.21 (d, 1H), 3.71-3.70 (d, 2H), 3.44-3.43 (t, 4H), 2.44-2.42 (t, 2H), 2.37-2.34 (t, 2H) 1.98-1.97 (s, 6H).

### Example 22

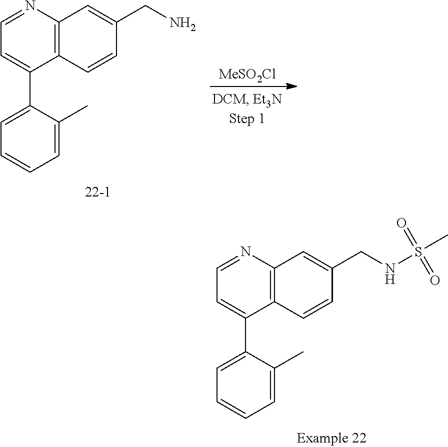 [SMILES: "MeSO2Cl" ; Cc1ccccc1-c1ccnc2cc(CN)ccc12 ; Cc1ccccc1-c1ccnc2cc(CNS(C)(=O)=O)ccc12]

Synthesis of N-((4-(o-tolyl)quinolin-7-yl)methyl)methanesulfonamide [Step 1]: To a stirred solution of [4-(o-tolyl)-7-quinolyl]methanamine (100 mg, 0.4 mmol) in DCM (2 mL) was added Et₃N (0.11 mL, 0.8 mmol) followed by methanesulfonyl chloride (0.05 mL, 0.6 mmol) at 0° C. and stirred at RT for 2 h. The reaction mixture was diluted with DCM and washed with water and brine, dried over Na₂SO₄ and concentrated under reduced pressure and purified via RP prep HPLC purification and lyophilized to afford N-((4-(o-tolyl)quinolin-7-yl)methyl)methanesulfonamide (14 mg). LC/MS. calculated for C₁₈H₁₈N₂O₂S: 326, found [M+H]⁺: 327; ¹H NMR (400 MHz, DMSO-d₆) δ 8.95 (d, 1H), 8.06 (S, 1H), 7.75 (br s, 1H), 7.53 (d, 1H), 7.44-7.43 (m, 2H), 7.39-7.37 (m, 3H), 7.23 (d, 1H), 4.38 (s, 2H), 2.92 (s, 3H), 1.97 (s, 3H).

### Example 23

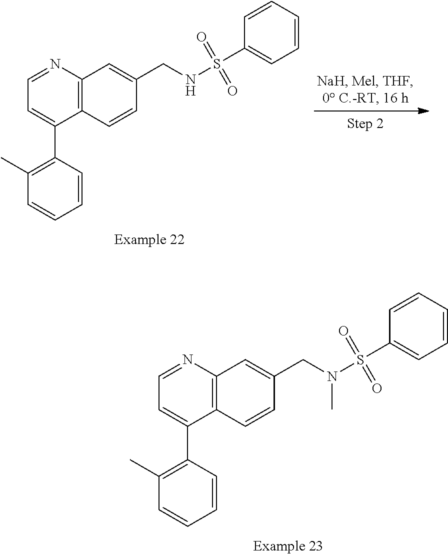 [SMILES: Cc1ccccc1-c1ccnc2cc(CNS(=O)(=O)c3ccccc3)ccc12 ; Cc1ccccc1-c1ccnc2cc(CN(C)S(=O)(=O)c3ccccc3)ccc12]

Synthesis of N-methyl-N-((4-(o-tolyl)quinolin-7-yl)methyl)benzenesulfonamide [step 2]: To a stirred solution of N-[[4-(o-tolyl)-7-quinolyl]methyl]benzenesulfonamide (60 mg, 0.15 mmol) in THF (2 mL) was added NaH, 60% in oil (25 mg, 0.6 mmol) at 0° C. and stirred for 30 min. Mel (0.037 mL, 0.60 mmol) was added and stirred at RT for 16 h. The reaction mixture was quenched with saturated aqueous ammonium chloride solution and extracted with ethyl acetate (thrice). The combined organic layer was washed with brine, dried over Na2SO4 and concentrated under reduced pressure. The crude was purified via RP prep HPLC purification and lyophilized to afford N-methyl-N-((4-(o-tolyl)quinolin-7-yl)methyl)benzenesulfonamide (12 mg). LC/MS: calculated for C₂₄H₂₂N₂O₂S: 402, found [M+H]⁺: 403; ¹H NMR (400 MHz, DMSO-d₆) δ 8.95 (d, 1H), 8.02 (s, 1H), 7.89 (d, 2H), 7.73 (t, 1H), 7.70-7.68 (m, 2H), 7.48 (d, 1H), 7.44-7.42 (m, 3H), 7.40-7.38 (m, 2H), 7.23 (d, 1H), 4.39 (s, 2H), 2.62 (s, 3H), 1.98 (s, 3H).

### Example 24

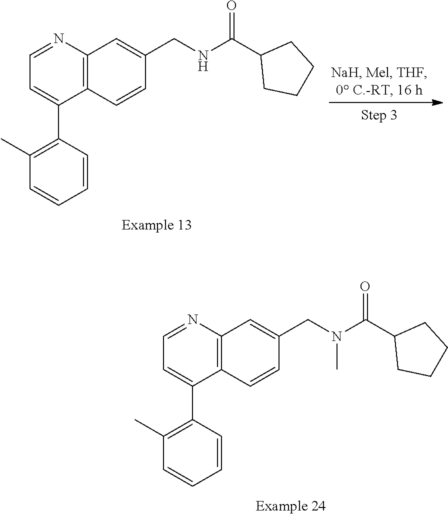 [SMILES: Cc1ccccc1-c1ccnc2cc(CNC(=O)C3CCCC3)ccc12 ; Cc1ccccc1-c1ccnc2cc(CN(C)C(=O)C3CCCC3)ccc12]

Synthesis of N-methyl-N-((4-(o-tolyl)quinolin-7-yl)methyl)cyclopentanecarboxamide [Step 3]: To a stirred solution of N-[[4-(o-tolyl)-7-quinolyl]methyl]cyclopentanecarboxamide (120 mg, 0.4 mmol) in THF (2 mL) was added NaH, 60% in oil (33 mg, 0.871 mmol) at 0° C. and stirred for 30 min. MeI (0.05 mL, 0.87 mmol) was added and stirred at RT for 16 h. The reaction mixture was quenched with saturated aqueous ammonium chloride solution and extracted with ethyl acetate (thrice). The combined organic layer was washed with brine, dried over Na₂SO₄ and concentrated under reduced pressure. The crude was purified via RP prep HPLC purification and lyophilized to afford N-methyl-N-[[4-(o-tolyl)-7-quinolyl]methyl]cyclopentanecarboxamide (8.5 mg). LC/MS: calculated for C₂₄H₂₆N₂O: 358, found [M+H]⁺: 359; ¹H NMR (400 MHz, DMSO-d₆) δ 8.94 (t, 1H), 7.83 (d, 1H), 7.42-7.35 (m, 6H), 7.23 (t, 1H), 4.87-7.67 (m, 2H), 3.11-3.08 (m, 1H), 3.01 (s, 3H), 1.98 (s, 3H), 1.86-1.82 (m, 2H), 1.74-1.55 (m, 5H), 1.48-1.23 (m, 1H).

### Example 25

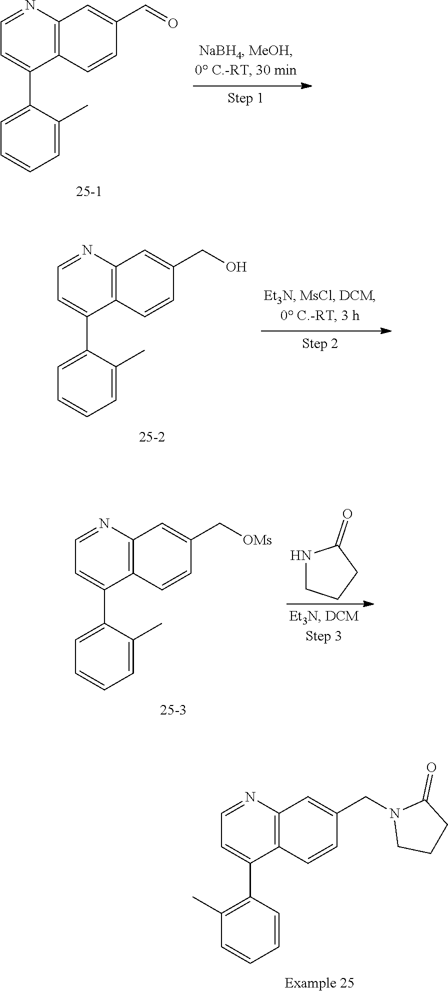 [SMILES: Cc1ccccc1-c1ccnc2cc(C=O)ccc12 ; Cc1ccccc1-c1ccnc2cc(CO)ccc12 ; Cc1ccccc1-c1ccnc2cc(COS(C)(=O)=O)ccc12 ; O=C1CCCN1 ; Cc1ccccc1-c1ccnc2cc(CN3CCCC3=O)ccc12]

Synthesis of (4-(o-tolyl)quinolin-7-yl)methanol [Step 1]: To a solution of 4-(o-tolyl)quinoline-7-carbaldehyde (150 mg, 0.60 mmol) in methanol (3 mL) was added NaBH₄ (70 mg, 1.8 mmol) at 0° C. and stirred at RT for 1 h. Solvent was evaporated under reduced pressure. The crude was partitioned between DCM and water. The organic layer was collected and further washed with water and brine, dried over Na₂SO₄ and concentrated under reduced pressure. The crude was purified via flash chromatography to get (4-(o-tolyl)quinolin-7-yl)methanol (80 mg). LCMS: Calculated for C₁₇H₁₅NO: 249, found [M+H]⁺: 250; ¹H NMR (400 MHz, DMSO-d₆) δ 8.93 (d, 1H), 8.02 (s, 1H), 7.49 (d, 1H), 7.42 (br s, 2H), 7.34 (br s, 3H), 7.23 (d, 1H), 5.42 (t, 1H), 4.70 (d, 2H), 1.95 (d, 3H).

Synthesis of (4-(o-tolyl)quinolin-7-yl)methyl methanesulfonate [Step 2]: To a stirred solution of (4-(o-tolyl)quinolin-7-yl)methanol (80 mg, 0.3 mmol) in DCM (4 mL) was added triethylamine (0.06 mL, 0.5 mmol) followed by methanesulfonyl chloride (75 mg, 0.6 mmol) at 0° C. drop wise. It was gradually warmed to RT and stirred for 3 h. The reaction mass was diluted with DCM and washed with water and brine, dried over Na₂SO₄ and concentrated under reduced pressure to get crude (4-(o-tolyl)quinolin-7-yl)methyl methanesulfonate (80 mg). The crude was used immediately for forwarding step.

Synthesis of 1-(4-(o-tolyl)quinolin-7-yl)methyl)pyrrolidin-2-one [Step 3]: To a stirred solution of (4-(o-tolyl)quinolin-7-yl)methyl methane sulfonate (80 mg, 0.3 mmol) in DMF (3 mL) was added NaH, 60% in oil (7.0 mg, 0.30 mmol) followed by pyrrlidin-2-one (20 mg, 0.3 mmol) at 0° C. It was gradually warmed to RT and stirred for 16 h. The reaction mixture was diluted with EtOAc, washed with cold water (twice) and brine, dried over anhydrous Na₂SO₄ and concentrated. The crude obtained was purified by flash column chromatography and lyophilized to afford 1-(4-o-tolyl)quinolin-7-yl-(methyl)pyrrolidin-2-one (20 mg). LCMS: Calculated for C₂₁H₂₀N₂O: 316, found [M+H]⁺: 317; ¹H NMR (400 MHz, DMSO-d₆) δ 8.94 (d, 1H), 7.91 (br s, 1H), 7.41 (d, 2H), 7.38-7.36 (m, 4H), 7.22 (d, 1H), 4.58 (d, 2H), 2.33 (t, 3H), 1.97-1.94 (m, 6H).

### Example 26

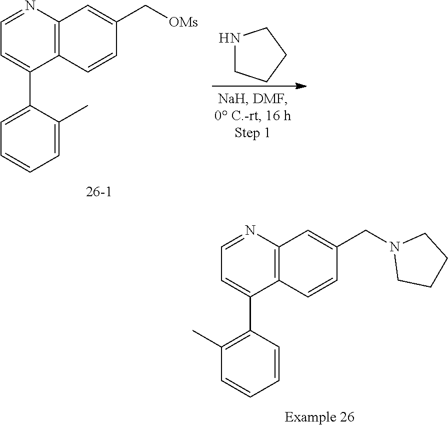 [SMILES: Cc1ccccc1-c1ccnc2cc(COS(C)(=O)=O)ccc12 ; C1CCNC1 ; Cc1ccccc1-c1ccnc2cc(CN3CCCC3)ccc12]

Synthesis of 7-(pyrrolidin-1-ylmethyl)-4-(o-tolyl)quinoline [Step 1]; To a stirred solution of [4-(o-tolyl)-7-quinolyl]methyl methanesulfonate (100 mg, 0.3 mmol) in DMF (3 mL) was added NaH, 60% in oil (8.8 mg, 0.4 mmol) followed by pyrrolidine (0.025 mL, 0.3 mmol) at 0° C. It was gradually warmed to RT and stirred for 16 h. It was quenched with saturated aqueous ammonium chloride solution and extracted with EtOAc. Organic layer was washed with cold water (thrice) and brine, dried over anhydrous Na₂SO₄ and concentrated. The crude was purified via RP prep HPLC purification and lyophilized to afford 7-(pyrrolidin-1-ylmethyl)-4-(o-tolyl)quinoline (30 mg). LCMS (ESI) Calcd. for C₂₁H₂₂N₂: 302, found [M+H]⁺=303; ¹H NMR (400 MHz, DMSO-d₆) δ 8.93-8.91 (d, 1H), 7.97 (s, 1H), 7.53-7.51 (d, 1H), 7.43-7.41 (m, 2H), 7.35-7.33 (m, 3H), 7.23-7.21 (d, 1H), 3.78 (s, 2H), 2.50 (s, 4H), 1.98 (s, 3H), 1.71 (s, 4H).

### Example 27

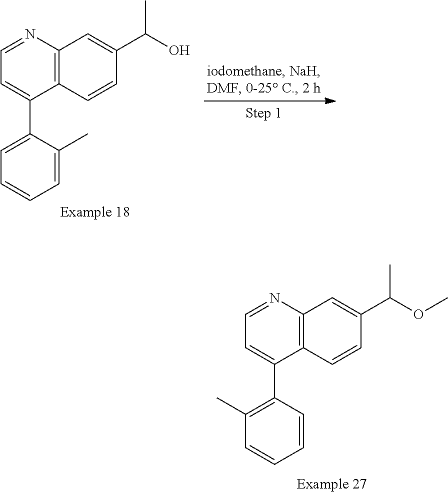

Synthesis of 7-(1-methoxyethyl)-4-(o-tolyl)quinoline [step 1]: To a suspension of 1-(4-(o-tolyl)quinolin-7-yl)ethan-1-ol (140 mg, 0.5 mmol) in DMF (3 mL) was added NaH (60% in oil) (32 mg, 0.8 mmol) at 0° C. After 30 min, iodomethane (0.04 mL, 0.6 mmol) was added and the reaction mixture was stirred at 25° C. for 2 h. The reaction mixture was quench with NH₄Cl and it was extracted with ethyl acetate (2×15 mL) washed with water and brine. The organic layer was evaporated under reduced pressure. The crude obtained was purified by RP prep-HPLC purification and lyophilized to afford 7-(1-methoxyethyl)-4-(o-tolyl)quinolone (66 mg). LCMS(ESI) Calcd. for C₁₉H₁₉NO: 277, found [M+H]⁺: 278; ¹H NMR (400 MHz, DMSO-d₆): δ 8.94 (d, 1H), 7.99 (s, 1H), 7.51 (d, 1H), 7.42-7.36 (m, 5H), 7.23 (d, 1H), 4.58-4.53 (m, 1H), 3.19 (s, 3H), 1.99 (s, 3H), 1.41 (d, 3H).

### Example 28

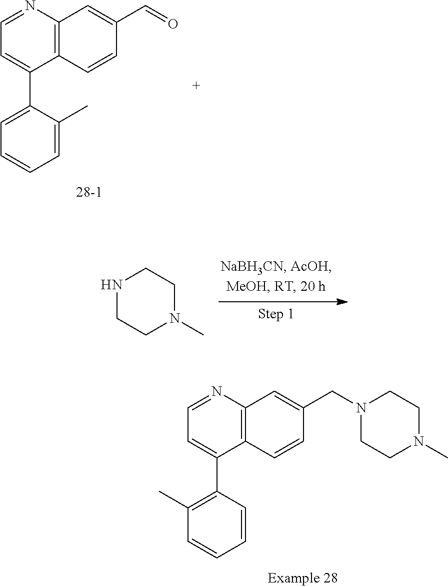

Synthesis of 7-((4-methylpiperazin-1-yl)methyl)-4-(o-tolyl)quinoline [Step 1]: To a solution of 4-(o-tolyl)quinoline-7-carbaldehyde (150 mg, 0.6 mmol) in methanol (2 mL) was added 1-methylpiperazine (60 mg, 0.60 mmol) followed by AcOH (0.05 mL) at RT and stirred for 16 h. Sodium cyanoborohydride (55 mg, 0.9 mmol) was added and stirred at RT for 4 h. The reaction mixture was concentrated under reduced pressure and diluted with EtOAc. It was washed with water and brine, dried over anhydrous sodium sulfate and concentrated. The crude was purified by RP prep HPLC purification and lyophilized to afford 7-((4-methylpiperazin-1-yl)methyl)-4-(o-tolyl)quinoline (14 mg). LCMS (ESI) Calcd. for C₂₂H₂₅N₃: 331, found [M+H]⁺=332; ¹H NMR (400 MHz, DMSO-d₆): δ 8.93-8.92 (d, 1H), 7.98 (s, 1H), 7.52-7.51 (d, 1H), 7.43-7.41 (m, 2H), 7.37-7.33 (m, 3H), 7.23-7.21 (d, 1H), 3.68 (s, 2H), 2.49 (m, 8H), 2.24 (s, 3H), 1.97 (s, 3H).

### Example 29-31

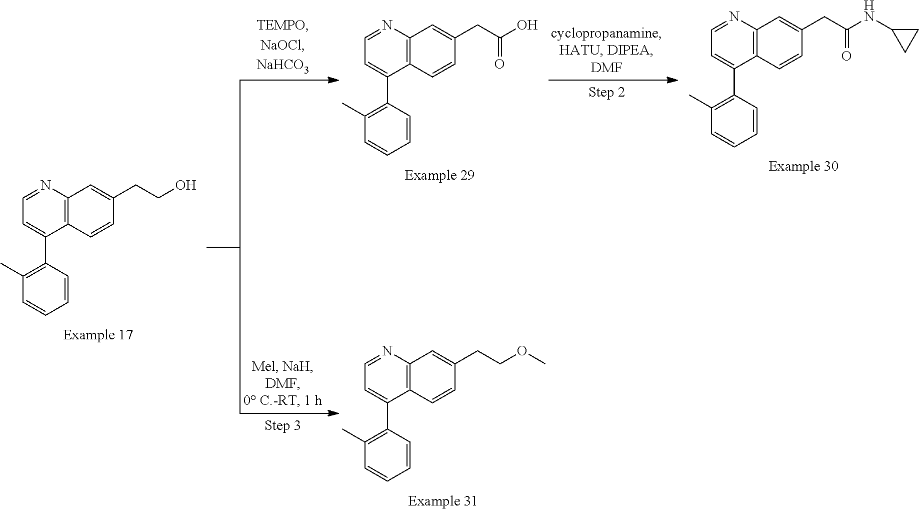

Synthesis of 2-(4-(o-tolyl)quinolin-7-yl)acetic acid [Step 1]: To a stirred solution of 2-(4-(o-tolyl)quinolin-7-yl)ethan-1-ol (350 mg, 1.3 mmol) in MeCN (18 mL) and water (18 mL) mixture, NaHCO₃ (170 mg, 2 mmol) and NaOCl(11-13%) (11 mL, 2.0 mmol) were added to the reaction mixture at 0° C. Then TEMPO (250 mg, 1.6 mmol) was added to the reaction mixture and it was heated at 60° C. for 16 h. After 16 h, it was filtered through celite bed and washed with 30% IPA in CHCl₃, concentrated under reduced pressure to get crude compound. The crude compound was purified by combi-flash column chromatography using 0-10% MeOH in DCM as a eluent to get 150 mg desired compound. The compound was again purified by RP Prep-HPLC purification to get 2-(4-(o-tolyl)quinolin-7-yl)acetic acid (20 mg). LCMS (ESI) Calcd. for C₁₈H₁₅NO₂: 277, found [M+H]⁺=278; ¹H NMR (400 MHz, DMSO-d₆) δ 8.92 (d, 1H), 7.96 (s, 1H), 7.50-7.38 (m, 3H), 7.41-7.28 (m, 3H), 7.22 (d, 1H), 3.76 (s, 2H), 1.98 (s, 3H).

Synthesis of N-cyclopropyl-2-(4-(o-tolyl)quinolin-7-yl)acetamide [Step 2]: To a stirred solution of 2-(4-(o-tolyl)quinolin-7-yl)acetic acid (80 mg, 0.3 mmol) and cyclopropanamine (80 μL, 1.1 mmol) in DMF (1 mL), DIPEA (0.2 mL, 1. mmol) was added at 0° C. Then HATU (330 mg, 0.9 mmol) was added to the reaction mixture and it was stirred at room temperature for 16 h. After 16 h, it was washed with water and extracted with ethyl acetate. Organic part was dried with Na₂SO₄, filtered and concentrated under reduced pressure to get crude compound. The crude compound was purified by RP Prep-HPLC purification to get N-cyclopropyl-2-(4-(o-tolyl)quinolin-7-yl)acetamide (13 mg). LCMS (ESI) Calcd. for C₂₁H₂₀N₂O: 316, found [M+H]⁺=317; ¹H NMR (400 MHz, DMSO-d₆) δ 892 (d, 1H), 8.24 (d, 1H), 7.95 (s, 1H), 7.47-7.38 (m, 3H), 7.40-7.28 (m, 3H), 7.21 (d, 1H), 3.59 (s, 2H), 2.72-2.56 (m, 1H), 1.98 (s, 3H), 0.65-0.57 (m, 2H), 0.45-0.36 (m, 2H).

Synthesis of 7-(2-methoxyethyl)-4-(o-tolyl)quinoline [Step 3]: To a stirred solution of NaH (18 mg, 0.5 mmol) in DMF (2 mL), 2-(4-(o-tolyl)quinolin-7-yl)ethan-1-ol (80 mg, 0.3 mmol) dissolved in DMF (1 ml), was added at 0° C. and it was stirred at 0° C. for 25 min. Then iodomethane (23 μL, 0.4 mmol) was added to the reaction mixture and it was stirred at same condition for 1 h. The reaction mixture was quenched with NH₄C, extracted with ethyl acetate, washed with brine solution. Organic part was dried with Na₂SO₄, filtered and concentrated under reduced pressure to get crude compound. The crude compound was purified by RP Prep-HPLC purification to get 7-(2-methoxyethyl)-4-(o-tolyl)quinoline (18 mg). LCMS (ESI) Calcd. for C₁₉H₁₉NO: 277, found [M+H]⁺=278; ¹H NMR (400 MHz, DMSO-d₆) δ 8.91 (d, 1H), 7.94 (s, 1H), 7.49-7.38 (m, 3H), 7.40-7.27 (m, 3H), 7.21 (d, 1H), 3.66 (t, 2H), 3.26 (s, 3H), 3.03 (t, 2H), 1.98 (s, 3H).

### Example 32

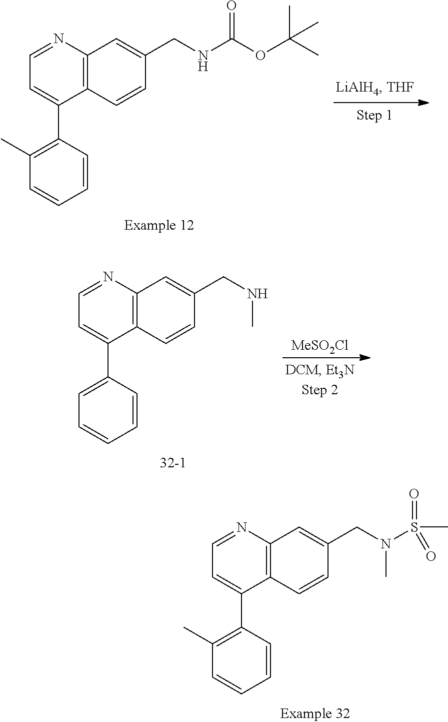

Synthesis of N-methyl-1-(4-(o-tolyl)quinolin-7-yl)methanamine [step-1]: A stirred solution tert-butyl ((4-(o-tolyl)quinolin-7-yl)methyl)carbamate (200 mg, 0.57 mmol) in THF (3 mL) was added LiAlH₄ (1.1 mL, 1.10 mmol) 1M solution in THF dropwise at 0° C. After addition, the reaction mixture was warmed up to RT and stirred for 4 h at 70° C. The reaction mixture was quenched with saturated Na₂SO₄ solution at 0° C. and extracted with ethyl acetate/methanol (9:1) and concentrated under reduced pressure to afford crude N-methyl-1-(4-(o-tolyl)quinolin-7-yl)methanamine (230 mg). LC/MS: calculated for C₁₈H₁₈N₂ [M]: 262, found [M+H]⁺: 263.

Synthesis of N-methyl-N-((4-(o-tolyl)quinolin-7-yl)methyl)methanesulfonamide [step-2]: A stirred solution of N-methyl-1-[4-(o-tolyl)-7-quinolyl]methanamine (110 mg, 0.42 mmol) in THF (2 mL) was added Et₃N (0.087 mL, 0.63 mmol) at RT and stirred for 30 min. MeSO₂Cl (0.060 mL, 0.63 mmol) was added to the reaction mixture and stirred at RT for 2 h. The reaction mixture was quenched with water and extracted with ethyl acetate. The Combined organic layer was washed with brine, dried over Na₂SO₄ and concentrated under reduced pressure. The crude was purified via prep HPLC purification to afford N-methyl-N-((4-(o-tolyl)quinolin-7-yl)methyl)methanesulfonamide (13 mg). ¹H NMR (400 MHz, DMSO-d₆) δ 8.97 (d, 1H), 8.04 (s, 1H), 7.50 (d, 1H), 7.44-7.38 (m, 5H), 7.23 (d, 1H), 4.46 (s, 2H), 3.01 (s, 3H), 2.73 (s, 3H), 1.98 (s, 3H). LC/MS: calculated for C₁₉H₂₀N₂O₂S [M]: 340, found [M+H]⁺: 341.

### Example 33

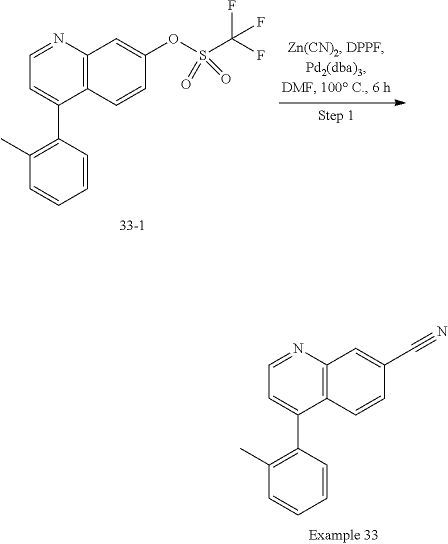 [SMILES: Cc1ccccc1-c1ccnc2cc(OS(=O)(=O)C(F)(F)F)ccc12 ; Cc1ccccc1-c1ccnc2cc(C#N)ccc12]

Synthesis of 4-(o-tolyl)quinoline-7-carbonitrile [Step 1]: A solution of 4-(0-tolyl)quinolin-7-yl trifluoromethanesulfonate (1 g, 2.7 mmol) in DMF (10 mL) was degassed for 10 minutes with argon balloon. Then zinc cyanide (320 mg, 2.7 mmol) was added followed by Pd₂(dba)₃ (230 mg, 0.25 mmol) and dppf (275 mg, 0.5 mmol). The mixture was heated at 100° C. under argon for 6 h. Crude LCMS showed desired product mass. Then the reaction mixture was filtered and volatiles were evaporated under reduced pressure. The crude was partitioned between ethyl acetate (50 ml) and ice cold water. The organic layer was washed with NaHCO₃ solution, dried with Na₂SO₄, concentrated to get the residue. The crude was purified through combiflash chromatography over silica gel using 35-40% ethyl acetate-hexanes as eluent afforded 4-(o-tolyl)quinoline-7-carbonitrile (450 mg). LCMS: calcd. for C₁₇H₁₂N₂: 244, found [M+H]⁺: 245; ¹H NMR (400 MHz, DMSO-d₆) δ 9.12 (d, 1H), 8.69 (d, 1H), 7.86 (d, 1H), 7.60 (d, 1H), 7.54 (d, 1H), 7.49-7.43 (m, 2H), 7.38 (t, 1H), 7.26 (d, 1H), 1.97 (s, 3H).

### Example 34-35

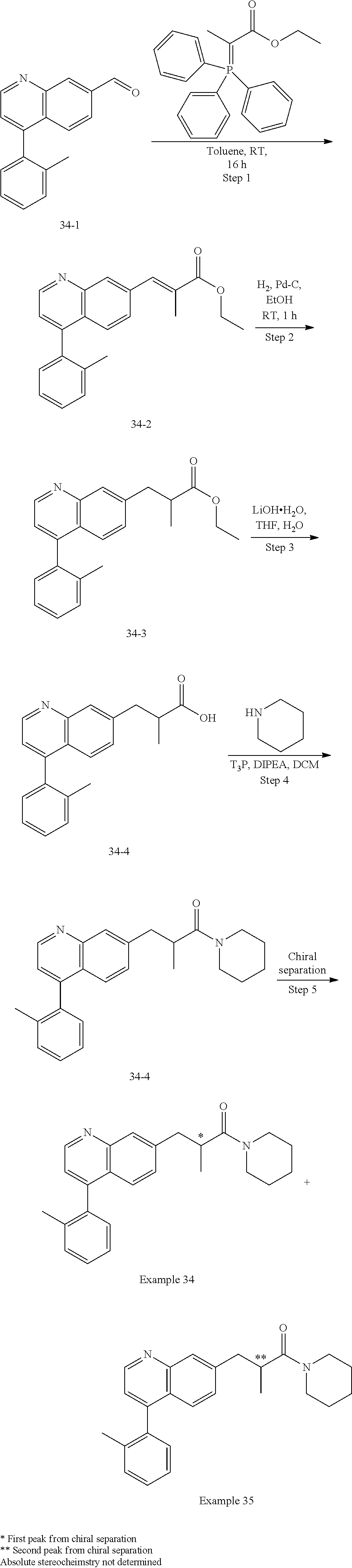

Synthesis of ethyl (E)-2-methyl-3-(4-(o-tolyl)quinolin-7-yl)acrylate [Step 1]: To a stirred solution of 4-(o-tolyl)quinoline-7-carbaldehyde (270 mg, 1.09 mmol) in Toluene (15 mL) was added ethyl 2-(triphenyl-λ5-phosphanylidene)propanoate (791 mg, 2.18 mmol) and stirred at RT for 5 h. Volatiles were removed under reduced pressure. It was partitioned between EtOAc and water. Organic layer was washed with brine, dried over Na₂SO₄ and concentrated under reduced pressure. The crude was purified by flash column chromatography and lyophilized to afford ethyl (L)-2-methyl-3-(4-(o-tolyl)quinolin-7-yl)acrylate (180 mg). LCMS (ESI): Calculated for C₂₂H₂₁NO₂: 331; found [M+H]⁺: 332; ¹H NMR (400 MHz, DMSO-d₆) δ 8.99 (d, 1H), 8.18 (s, 1H), 7.79 (s, 1H), 7.64 (d, 1H), 7.43-7.41 (m, 4H), 7.39-7.37 (m, 1H), 7.26 (d, 1H), 4.22 (q, 2H), 2.15 (s, 3H), 1.99 (s, 3H), 1.30 (t, 3H).

Synthesis of ethyl 2-methyl-3-(4-(o-tolyl)quinolin-7-yl)propanoate [Step 2]: To a stirred solution of ethyl (E)-2-methyl-3-(4-(o-tolyl)quinolin-7-yl)acrylate (160 mg, 0.483 mmol) in Ethanol (5 mL) was added Pd—C(10%) (15 mg, 0.483 mmol) and hydrogenated under hydrogen balloon pressure at RT for 1 h. Reaction mixture was filtered through celite bed and purified by combiflash to afford ethyl 2-methyl-3-(4-(o-tolyl)quinolin-7-yl)propanoate (120 mg). LCMS (ESI): Calculated for C₂₂H₂₃NO₂: 333, found [M+H]⁺=334.

Synthesis of 2-methyl-3-(4-(o-tolyl)quinolin-7-yl)propanoic acid [Step 3]: To a stirred solution of ethyl 2-methyl-3-(4-(o-tolyl)quinolin-7-yl)propanoate (120 mg, 0.360 mmol) in THF (13 mL) and Water (3 mL) was added LiOH·H₂O (45 mg, 1.08 mmol) and stirred at RT for 4 h. Volatiles were removed under reduced pressure and partitioned between EtOAc and water. Aqueous layer was acidified with 10% aqueous citric acid solution and extracted with EtOAc (thrice). Combined organic layer was washed with brine, dried over Na₂SO₄ and concentrated under reduced pressure to afford 2-methyl-3-(4-(o-tolyl)quinolin-7-yl)propanoic acid (80 mg). LCMS (ESI): Calculated for C₂₀H₁₉NO₂: 305, found [M+H]⁺=306; ¹H NMR (400 MHz, DMSO-d₆) δ 12.17 (br s, 1H), 8.89-8.88 (d, 1H), 7.87 (s, 1H), 7.40-7.38 (m, 3H), 7.33-7.26 (m, 3H), 7.20-7.18 (d, 1H), 3.10-3.05 (m, 1H), 2.85-2.74 (m, 2H), 1.95 (s, 3H), 1.07-1.06 (s, 3H).

Synthesis of 2-methyl-1-(piperidin-1-yl)-3-(4-(o-tolyl)quinolin-7-yl)propan-1-one [Step 4]: To a stirred solution of 2-methyl-3-[4-(o-tolyl)-7-quinolyl]propanoic acid (100 mg, 0.327 mmol) and piperidine (0.05 mL, 0.5 mmol) in DCM (4 mL) was added DIPEA (0.17 mL, 1 mmol) followed by T3P (0.35 mL, 0.5 mmol) at 0° C. and stirred at RT for 2 h. The reaction mass was diluted with DCM and washed with water and brine, dried over Na2SO4 and concentrated under reduced pressure. The crude was purified by flash chromatography to afford 2-methyl-1-(piperidin-1-yl)-3-(4-(o-tolyl)quinolin-7-yl)propan-1-one (100 mg). The racemic mixture was separated via chiral separation.

Synthesis of chiral analogs of 2-methyl-1-(piperidin-1-yl)-3-(4-(o-tolyl)quinolin-7-yl)propan-1-one [Step 5]: 2-methyl-1-(piperidin-1-yl)-3-(4-(o-tolyl)quinolin-7-yl)propan-1-one (100 mg) was purified by chiral prep HPLC purification and lyophilized to afford Example 34, Peak 1, as 2-methyl-1-(piperidin-1-yl)-3-(4-(o-tolyl)quinolin-7-yl)propan-1-one (30 mg) and Example 35, Peak 2 as 2-methyl-1-(piperidin-1-yl)-3-(4-(o-tolyl)quinolin-7-yl)propan-1-one (45 mg). Chirality of both enantiomers was not determined.

Example 34: 2-methyl-1-(piperidin-1-yl)-3-(4-(o-tolyl)quinolin-7-yl)propan-1-one (peak-1): LCMS (ESI) Calculated for C₂₅H₂₈N₂O:372, found [M+H]+: 373; ¹H NMR (400 MHz, DMSO-d₆) δ 8.91-8.90 (d, 1H), 7.89-7.85 (d, 1H), 7.43-7.40 (m, 3H), 7.39-7.33 (m, 1H), 7.30-7.28 (m, 2H), 7.19-7.14 (m, 1H), 3.54 (m, 1H), 3.38-3.22 (m, 3H), 3.15 (m, 1H), 3.07-3.05 (m, 1H), 2.82-2.76 (m, 1H), 1.95-1.94 (d, 3H), 1.42-1.30 (m, 5H), 1.23-1.14 (m, 1H), 1.07-1.05 (d, 3H).

Example 35: 2-methyl-1-(piperidin-1-yl)-3-(4-(o-tolyl)quinolin-7-yl)propan-1-one (peak-2): LCMS (ESI): calculated for C₂₅H₂₈N₂O: 372, found [M+H]+: 373; ¹H NMR (400 MHz, DMSO-d₆) δ 8.91-8.90 (d, 1H), 7.89-7.85 (d, 1H), 7.43-7.40 (m, 3H), 7.39-7.33 (m, 1H), 7.30-7.28 (m, 2H), 7.19-7.14 (m, 1H), 3.54 (m, 1H), 3.38-3.22 (m, 3H), 3.15 (m, 1H), 3.07-3.05 (m, 1H), 2.82-2.76 (m, 1H), 1.95-1.94 (d, 3H), 1.42-1.30 (m, 5H), 1.23-1.14 (m, 1H), 1.07-1.05 (d, 3H).

PREP-HPLC chiral method: Thar SFC-80 series instrument by using Lux i-AMYLOSE-3 column (250×21.2 mm),5μ, operating at 35° C. temperature, maintaining flow rate of 60 gm/min, using 50% CO2 in super critical state & 50% of 0.3% Isopropylamine in MEOH as mobile phase, held this isocratic mixture upto 18 min and also maintained the isobaric condition of 100 bar at 220 nm wavelength.

### Example 36

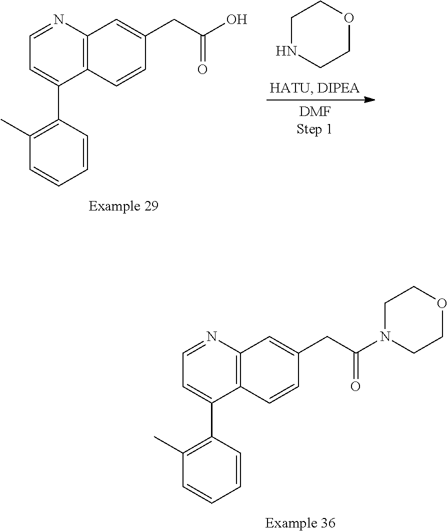 [SMILES: Cc1ccccc1-c1ccnc2cc(CC(=O)O)ccc12 ; C1COCCN1 ; Cc1ccccc1-c1ccnc2cc(CC(=O)N3CCOCC3)ccc12]

Synthesis of 1-morpholino-2-(4-(o-tolyl)quinolin-7-yl)ethan-1-one [Step 1]: To a stirred solution of 2-(4-(o-tolyl)quinolin-7-yl)acetic acid (110 mg, 0.4 mmol) and morpholine (0.14 mL, 1.6 mmol) in DMF (1 mL), was added DIPEA (0.22 mL, 1.6 mmol) followed by HATU (452 mg, 1.2 mmol) at 0° C. and stirred at rt for 16 h. Reaction mixture was diluted with EtOAc and washed with water and brine, dried over anhydrous sodium sulfate and concentrated in vacuo. The crude was purified by prep HPLC purification and lyophilized to afford 1-morpholino-2-(4-(o-tolyl)quinolin-7-yl)ethan-1-one (50 mg). LCMS (ESI) Calcd. for C22H22N2O2: 346, found [M+H]+=347; ¹H NMR (400 MHz, DMSO-d₆): δ 8.93-8.92 (d, 1H), 7.94 (d, 1H), 7.44-7.40 (m, 3H), 7.38-7.32 (m, 3H), 7.26-7.24 (d, 1H), 3.97 (s, 2H), 3.55 (m, 6H), 3.48-3.46 (d, 2H), 1.98 (s, 3H).

### Example 37-38

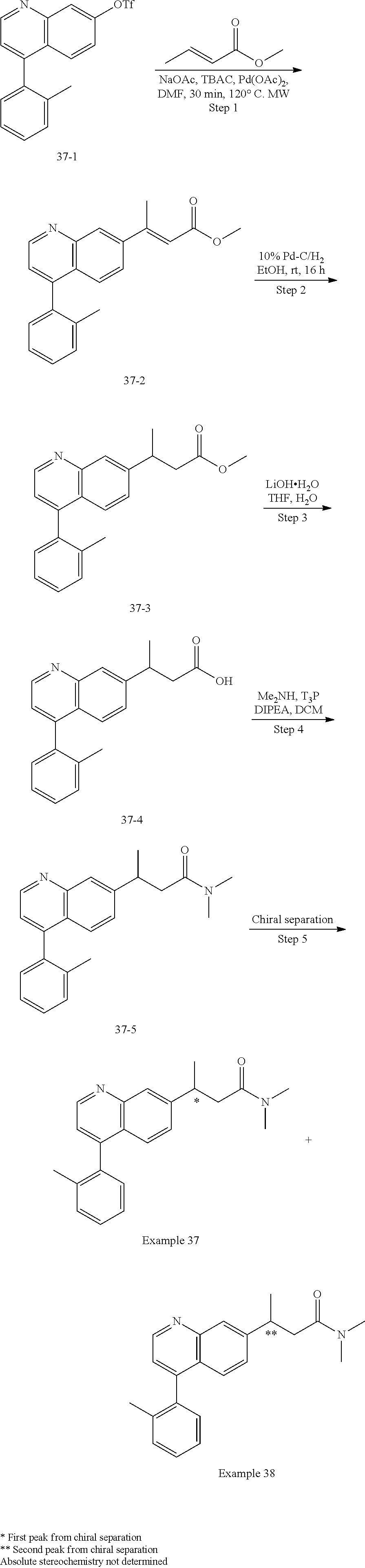

Synthesis of methyl (E)-3-(4-(o-tolyl)quinolin-7-yl)but-2-enoate [Step 1]: To a stirred solution of 4-(o-tolyl)quinolin-7-yl trifluoromethanesulfonate (1.0 g, 2.7 mmol) in DMF (20 mL) was added methyl (E)-but-2-enoate (0.6 mL, 5.4 mmol) and the reaction mixture was purged with argon gas for 5 min. Then tetrabutylammonium chloride (1.5 g, 5.4 mmol), NaOAc (450 mg, 5.4 mmol) and Pd(OAc)₂ (60 mg, 0.3 mmol) were added to the solution and the reaction mixture was irradiated at 120° C. in microwave for 45 min. The reaction mixture was filtered through celite bed and the filtrate was diluted with EtOAc and washed with water, brine, dried over Na₂SO₄ and concentrated to get the crude compound. The crude was purified via flash column chromatography to afford methyl (E)-3-(4-(o-tolyl)quinolin-7-yl)but-2-enoate (400 mg). LC/MS. calculated for C₂₁H₁₉NO₂: 317, [M+H]⁺: 318. ¹H NMR (400 MHz, DMSO-d₆) δ 9.00 (d, 1H), 8.26 (d, 1H), 7.80-7.77 (dd, 1H), 7.45-7.34 (m, 4H), 7.25 (d, 2H), 6.38 (d, 1H), 3.70 (s, 3H), 2.65 (d, 3H), 1.99 (s, 3H).

Synthesis of methyl 3-(4-(o-tolyl)quinolin-7-yl)butanoate [Step 2]: A stirred solution of methyl (E)-3-(4-(o-tolyl)quinolin-7-yl)but-2-enoate (200 mg, 0.6 mmol) in ethanol (5 mL) was purged with argon gas for 5 min followed by 10% Pd/C (20 mg, 0.6 mmol) and the reaction mixture was hydrogenated using hydrogen balloon at RT for 16 h. The reaction mixture was filtered through celite bed and the filtrate was concentrated to get the crude compound. The crude was purified by flash column chromatography to afford methyl 3-(4-(o-tolyl)quinolin-7-yl)butanoate (190 mg). LC/MS: calculated for C₂₁H₂₁NO₂: 319, found [M+H]⁺: 320; ¹H NMR (400 MHz, DMSO-d₆) δ 8.92 (d, 1H), 7.93 (s, 1H), 7.49 (d, 1H), 7.42-7.41 (m, 2H), 7.34-7.30 (m, 3H), 7.21 (d, l H), 3.59 (s, 3H), 3.53-3.50 (m, 1H), 2.80-2.68 (m, 2H), 1.90 (s, 3H), 1.15 (d, 3H).

Synthesis of 3-(4-(o-tolyl)quinolin-7-yl)butanoic acid [Step 3]: To a stirred solution of methyl 3-(4-(o-tolyl)quinolin-7-yl)butanoate (200 mg, 0.6 mmol) in THF (8 mL) and water (3 mL) mixture, LiOH·H₂O (75 mg, 1.9 mmol) was added and stirred at RT for 4 h. The reaction mixture was acidified with 1(N) HCl and partitioned between EtOAc and water. The organic layer was collected and the aqueous layer was further extracted with EtOAc (twice). The combined organic layer was washed with brine, dried over anhydrous sodium sulfate and concentrated under reduced pressure to afford 3-(4-(o-tolyl)quinolin-7-yl)butanoic acid (150 mg). The crude was used for next step without further purification. LC/MS: calculated for C₂₀H₁₉NO₂: 305, [M+H]⁺: 306; ¹H NMR (400 MHz, DMSO-d₆) δ 12.06 (br s, 1H), 8.91 (d, 1H), 7.92 (s, 1H), 7.50 (d, 1H), 7.43-7.40 (m, 2H), 7.36-7.32 (m, 3H), 7.31 (d, 11-), 3.40-3.36 (m, 1H), 2.70-2.62 (m, 2H), 1.98 (s, 3H) 1.32 (d, 3H).

Synthesis of N,N-dimethyl-3-(4-(o-tolyl)quinolin-7-yl)butanamide [Step 4]: To a stirred solution of 3-(4-(0-tolyl)quinolin-7-yl)butanoic acid (250 mg, 0.8 mmol) in DCM (5 mL) was added dimethyl amine hydrochloride (200 mg, 2.5 mmol), DIPEA (0.3 mL, 2.5 mmol) and the reaction mixture was cooled to 0° C. Into this cooled solution was added T₃P (50% in EtOAc) (1.4 mL, 2.5 mmol) and the reaction mixture was allowed to warm up to RT and stirred for 16 h. The reaction mixture was diluted with DCM and washed with water, brine, dried over Na₂SO₄ and concentrated to get the crude. The crude was purified by flash chromatography to afford N,N-dimethyl-3-(4-(o-tolyl)quinolin-7-yl)butanamide (152 mg) as a solid. The two enantiomers were separated by chiral SFC HPLC method.

Synthesis of chiral analogs of N,N-dimethyl-3-(4-(o-tolyl)quinolin-7-yl)butanamide [Step 5]: The racemic N,N-dimethyl-3-(4-(o-tolyl)quinolin-7-yl)butanamide (152 mg) was separated via SFC chiral prep HPLC purification and lyophilized to afford Example 37, Peak 1, as N,N-dimethyl-3-(4-(o-tolyl)quinolin-7-yl)butanamide (57 mg) as solid and Example 38, Peak 2, as N,N-dimethyl-3-(4-(o-tolyl)quinolin-7-yl)butanamide (51 mg). Absolute stereochemistry of both the enantiomers was not determined.

Example 37, Peak 1: NN-dimethyl-3-(4-(o-tolyl)quinolin-7-yl)butanamide: LC/MS: calculated for C₂₂H₂₄N₂O: 332, found [M+H]⁺: 333; ¹H NMR (400 MHz, DMSO-d₆) 8.91 (d, 1H), 7.93 (s, 1H), 7.51-7.49 (m, 1H), 7.43-7.40 (m, 2H), 7.37-7.32 (m, 1H), 7.31-7.29 (m, 1H), 7.21 (d, 2H), 3.42-3.39 (m, 1H), 2.95 (s, 3H), 2.77 (s, 3H), 2.74-2.69 (m, 1H), 2.68-2.63 (m, 1H), 1.98 (s, 3H), 1.29 (d, 3H).

Example 38, Peak 2: N,N-dimethyl-3-(4-(o-tolyl)quinolin-7-yl)butanamide: LC/MS: calculated for C₂₂H₂₄N₂O: 332.2, [M+H]⁺: 333.3. HPLC: Rt=6.72 min (98%); Chiral HPLC: Rt=5.36 min 98% ee. ¹H NMR (400 MHz, DMSO-d₄) 8.91 (d, 1H), 7.93 (s, 1H), 7.51-7.48 (m, 1H), 7.43-7.40 (m, 2H), 7.36-7.34 (m, 3H), 7.33-7.32 (m, 1H), 3.40-3.39 (m, 1H), 2.95 (s, 3H), 2.78 (s, 3H), 2.74-2.50 (m, 1H), 2.68-2.64 (m, 1H), 1.98 (s, 3H), 1.29 (d, 3H).

SFC chiral prep HPLC method: Column: (R,R) WHELK-01 (21 mm×250 mm), 5p Flow: 50 g/min, Mobile Phase:80% CO₂₊₂₀% (0.3% IPamine in Methanol), ABPR: 100 bar, Temp: 35° C., UV: 230 nm, Diluent: Methanol+DCM.

### Example 39

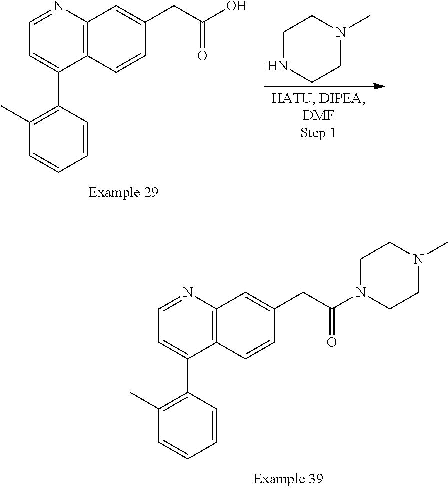

Synthesis of 1-(4-methylpiperazin-1-yl)-2-(4-(o-tolyl)quinolin-7-yl)ethan-1-one [Step 1]: To a stirred solution of 2-(4-(o-tolyl)quinolin-7-yl)acetic acid (120 mg, 0.4 mmol) and 1-methylpiperazine (175 mg, 1.7 mmol) in DMF (1 mL) was added DLPEA (0.24 mL, 1.7 mmol) followed by HATU (495 mg, 1.30 mmol) at 0° C. and stirred at rt for 16 h. The reaction mixture was diluted with EtOAc and washed with water (thrice) and brine, dried over anhydrous sodium sulfate and concentrated. The crude was purified via RP prep HPLC purification and lyophilized to afford 1-(4-methylpiperazin-1-yl)-2-(4-(o-tolyl)quinolin-7-yl)ethan-1-one (55 mg). LCMS (ESI) Calcd. for C₂₃H₂₅N₃O: 359, found [M+H]⁺=360; ¹H NMR (400 MHz, DMSO-d₆): δ 8.93-8.92 (d, 1H), 7.94 (d, 1H), 7.45-7.31 (m, 6H), 7.26-7.24 (d, 1H), 3.96 (s, 2H), 3.58-3.51 (d, 4H), 2.42 (br s, 4H), 2.27 (s, 3H), 1.99-1.98 (s, 3H). Peak at 8.13 (s, 0.6H) is due to formic acid.

### Example 40

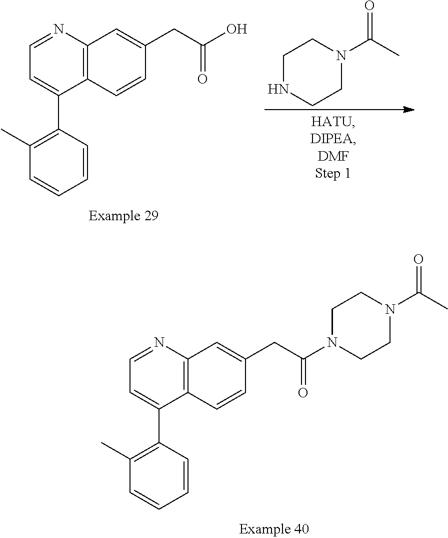

Synthesis of 1-(4-acetylpiperazin-1-yl)-2-(4-(o-tolyl)quinolin-7-yl)ethan-1-one [Step 1]: To a stirred solution of 2-(4-(o-tolyl)quinolin-7-yl)acetic acid (150 mg, 0.5 mmol) and 1-(piperazin-1-yl)ethan-1-one (275 mg, 2.2 mmol) in DMF (1 mL), was added DIPEA (0.3 mL, 2.2 mmol) followed by HATU (615 mg, 1.6 mmol) at 0° C. and stirred at RT for 16 h. The reaction mixture was diluted with EtOAc and washed with aqueous 5% K₂CO₃ solution, water and brine, dried over anhydrous sodium sulfate and concentrated in vacuo. The crude was purified by RP prep HPLC purification and lyophilized to afford 1-(4-acetylpiperazin-1-yl)-2-(4-(o-tolyl)quinolin-7-yl)ethan-1-one (53 mg). LCMS (ESI) Calcd. for C₂₄H₂₅N₃O₂: 387, found [M+H]⁺=388; ¹H NMR (400 MHz, DMSO-d₆): δ 8.93-8.92 (d, 1H), 7.94 (d, 1H), 7.42-7.40 (m, 3H), 7.37-7.31 (m, 3H), 7.23-7.21 (d, 1H), 4.0-3.99 (d, 2H), 3.58-3.53 (m, 3H), 3.42 (m, 5H), 2.0-1.96 (d, 6H).

### Example 41

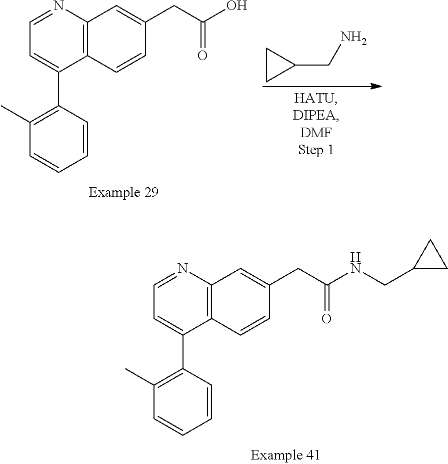 [SMILES: Cc1ccccc1-c1ccnc2cc(CC(=O)O)ccc12 ; NCC1CC1 ; Cc1ccccc1-c1ccnc2cc(CC(=O)NCC3CC3)ccc12]

Synthesis of N-(cyclopropylmethyl)-2-(4-(o-tolyl)quinolin-7-yl)acetamide [Step 1]: To a stirred solution of 2-(4-(o-tolyl)quinolin-7-yl)acetic acid (120 mg, 0.4 mmol) and cyclopropylmethanamine (125 mg, 1.7 mmol) in DMF (1 mL), was added DIPEA (0.25 mL, 1.7 mmol) followed by HATU (495 mg, 1.3 mmol) at 0° C. and stirred at rt for 16 h. The reaction mixture was diluted with EtOAc and washed with water and brine, dried over anhydrous sodium sulfate and concentrated in vacuo. The crude was purified by RP prep HPLC purification and lyophilized to afford N-(cyclopropylmethyl)-2-(4-(o-tolyl)quinolin-7-yl)acetamide (27 mg). LCMS (ESI) Calcd. for C₂₂H₂₂N₂O: 330, found [M+H]⁺=331; ¹H NMR (400 MHz, DMSO-d₆): δ 8.93-8.92 (d, 1H), 8.26-8.24 (t, 1H), 7.97 (s, 1H), 7.46-7.37 (m, 3H), 7.36-7.30 (m, 3H), 7.22-7.20 (d, 1H), 3.64 (s, 2H), 2.96-2.93 (t, 2H), 1.97 (s, 3H), 0.91-0.86 (m, 1H), 0.42-0.38 (m, 2H), 0.21-0.19 (t, 2H).

### Example 42

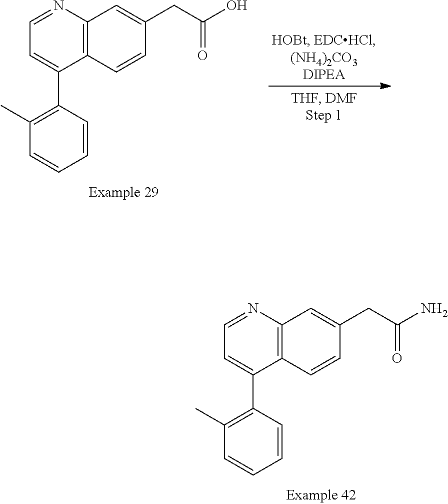 [SMILES: Cc1ccccc1-c1ccnc2cc(CC(=O)O)ccc12 ; Cc1ccccc1-c1ccnc2cc(CC(N)=O)ccc12]

Synthesis of 2-(4-(o-tolyl)quinolin-7-yl)acetamide [step-1]: To a stirred solution of 2-(4-(o-tolyl)quinolin-7-yl)acetic acid (120 mg, 0.4 mmol) in THF (3 mL) and DMF (0.8 mL) were added HOBt (90 mg, 0.6 mmol), EDC·HCl (125 mg, 0.6 mmol) and DIPEA (0.24 mL, 1.7 mmol) followed by (NH4)₂CO₃(165 mg, 1.7 mmol) and stirred at RT for 17 h. The reaction mixture was quenched with water and extracted with ethyl acetate. The combined organic layer was washed with brine, dried over Na₂SO₄ and concentrated under reduced pressure. The crude was purified via RP prep HPLC purification and lyophilized to afford 2-(4-(o-tolyl)quinolin-7-yl)acetamide (24 mg). LC/MS: calculated for C₁₈H₁₆N₂O: 276, found [M+H]⁺: 277; ¹H NMR (400 MHz, DMSO-d₆) δ 8.93 (d, 1H), 7.97 (s, 1H), 7.58 (s, 1H), 7.46-7.40 (m, 3H), 7.37-7.22 (m, 3H), 7.21 (d, 1H), 6.97 (s, 1H), 3.61 (s, 2H), 1.98 (s, 3H).

### Example 43-44

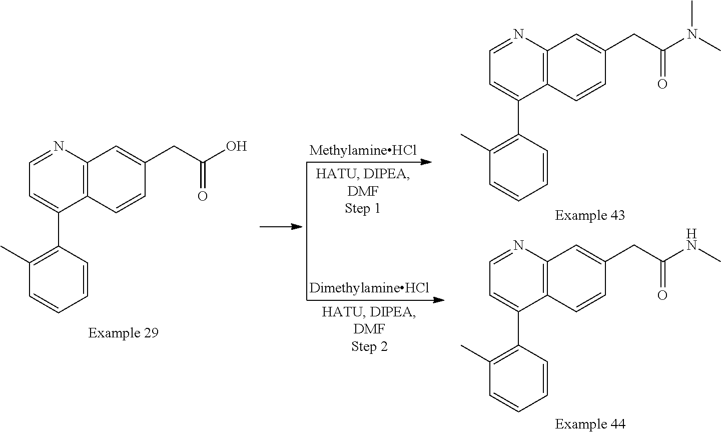

Synthesis of N,N-dimethyl-2-(4-(o-tolyl)quinolin-7-yl)acetamide [Step 1]: To a stirred solution of 2-(4-(o-tolyl)quinolin-7-yl)acetic acid (100 mg, 0.4 mmol) and Dimethylamine hydrochloride (29 mg, 0.4 mmol) in DMF (1 mL), DLPEA (0.24 mL, 1.7 mmol) was added at 0° C. Then HATU (493 mg, 1.3 mmol) was added to the reaction mixture and it was stirred at rt for 16 h. Reaction mixture was diluted with ethyl acetate, washed with water and brine solution, dried over Na2SO4 and evaporated under reduced pressure, crude was purified by Prep HPLC purification and lyophilized to get N,N-dimethyl-2-(4-(o-tolyl)quinolin-7-yl)acetamide (25 mg); ¹H NMR (400 MHz, DMSO-d₆) δ 8.98 (d, 1H), 7.94 (s, 1H), 7.46-7.34 (m, 6H), 7.24 (d, 1H), 3.95 (s, 2H), 3.06 (s, 3H), 2.85 (s, 3H), 1.99 (s, 3H). LC/MS: calculated for C20H20N2O: 304, found [M+H]=305.

Synthesis of N-methyl-2-(4-(o-tolyl)quinolin-7-yl)acetamide [Step 2]: To a stirred solution of 2-(4-(o-tolyl)quinolin-7-yl)acetic acid (100 mg, 0.4 mmol) and methylamine hydrochlide (24 mg, 0.4 mmol) in DMF (1 mL), DIPEA (0.24 mL, 1.7 mmol) was added at 0° C. Then HATU (493 mg, 1.3 mmol) was added to the reaction mixture and it was stirred at rt for 16 h. Reaction mixture was diluted with ethyl acetate, washed with water and brine solution, dried over Na2SO4 and evaporated under reduced pressure, crude was purified by Prep HPLC purification and lyophilised to get N-methyl-2-(4-(o-tolyl)quinolin-7-yl)acetamide (20 mg). ¹H NMR (400 MHz, DMSO-d₆) δ 8.93 (d, 1H), 8.05 (s, 1H), 7.96 (s, 1H), 7.45-7.41 (m, 3H), 7.35-7.30 (m, 3H), 7.22 (d, 1H), 3.62 (s, 2H), 2.59 (s, 3H), 1.97 (s, 3H). LC/MS. calculated for C19H18N2O: 290, found [M+H]=291.

### Example 45-47

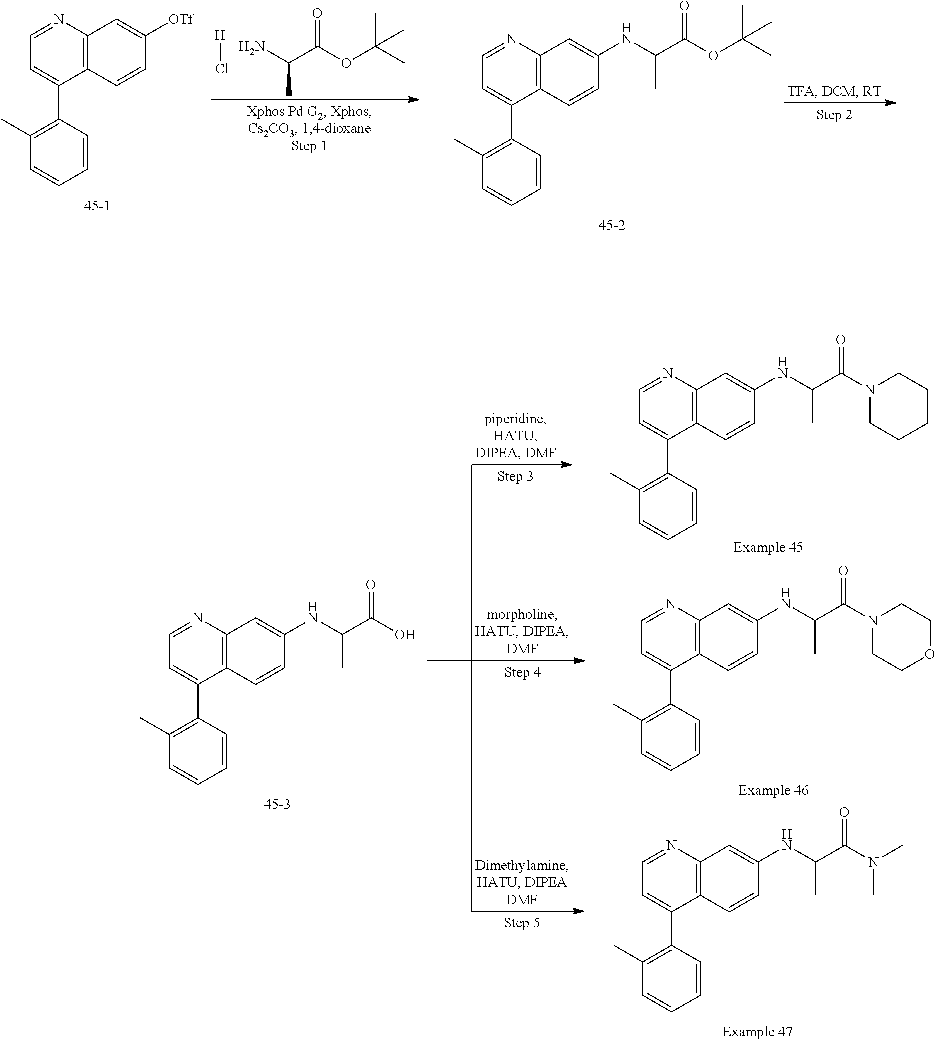

Synthesis of tert-butyl (4-(o-tolyl)quinolin-7-yl)alaninate [Step 1]: In a sealed tube 4-(o-tolyl)quinolin-7-yl trifluoromethanesulfonate (500 mg, 1.4 mmol) was taken in 1,4-dioxane (5 mL) and bubbled with nitrogen gas for 10 min. Then Xphos Pd G2 (75 mg, 0.1 mmol), Xphos (85 mg, 0.2 mmol) and Cs₂CO.; (665 mg, 2.0 mmol) were added and nitrogen purging was continued for another 10 min. Finally tert-butyl D-alaninate hydrochloride (270 mg, 1.5 mmol) was added and the reaction mixture was heated at 100° C. for 24 h. The reaction mixture was filtered and purified by column chromatography over silica gel using EtOAc and hexanes to yield tert-butyl (4-(o-tolyl)quinolin-7-yl)alaninate (110 mg). LCMS (ESI): calculated for C₂₃H₂₆N₂O₂, 362, found [M+H]⁺: 363. The chirality was lost due to heating in high temperature.

Synthesis of (4-(o-tolyl)quinolin-7-yl)alanine [Step 2]: To a stirred solution of tert-butyl (4-(o-tolyl)quinolin-7-yl)alaninate (250 mg, 0.7 mmol) in DCM (1 mL), TFA (0.5 mL, 6.5 mmol) was added and the reaction mixture was stirred at RT for 16 h. The volatiles were evaporated to yield (4-(o-tolyl)quinolin-7-yl)alanine (200 mg). LCMS (ESI): calculated for C₁₉H₁₈N₂O₂, 306, found [M+H]⁺: 307.

Synthesis of 1-(piperidin-1-yI)-2-((4-(o-tolyl)quinolin-7-yl)amino)propan-1-one [Step 3]: To a stirred solution of (4-(o-tolyl)quinolin-7-yl)alanine (100 mg, 0.3 mmol) in DMF (1 mL), HATU (250 mg, 0.7 mmol), and N,N-Diisopropylethylamine (0.3 mL, 1.6 mmol) were added at 0° C. and the reaction mixture was stirred for 30 min. Then morpholine (0.04 mL, 0.5 mmol) was added and the reaction mixture was stirred at RT for 16 h. The reaction mixture was diluted with EtOAc and washed with 5% aq. Na₂CO₃, water and brine. It was dried over Na₂SO₄, filtered and evaporated to yield brown gum. The crude was purified by RP prep HPLC purification and lyophilized to yield 1-(piperidin-1-yl)-2-((4-(o-tolyl)quinolin-7-yl)amino)propan-1-one (10 mg). LCMS (ESI): calculated for C₂₄H₂₇N₃O, 373, found [M+H]⁺: 374; ¹H NMR (400 MHz, DMSO-d₆) δ 8.66 (d, 1H), 7.37-7.30 (m, 3H), 7.18-7.16 (m, 1H), 7.05 (br s, 2H), 6.94 (d, 1H), 6.87 (d, 1H), 6.45 (d, 1H), 4.66-4.62 (m, 1H), 3.66 (br s, 1H), 3.57-3.53 (m, 2H), 3.39-3.32 (m, 1H), 1.98 (s, 3H), 1.62 (br s, 4H), 4.44 (br s, 2H), 1.31 (d, 3H).

Synthesis of 1-morpholino-2-((4-(o-tolyl)quinolin-7-yl)amino)propan-1-one [Step 4]: To a stirred solution of (4-(o-tolyl)quinolin-7-yl)alanine (100 mg, 0.3 mmol) in DMF (1 mL), HATU (250 mg, 0.7 mmol), and N,N-Diisopropylethylamine (0.3 mL, 1.6 mmol) were added at 0° C. and the reaction mixture was stirred for 30 min. Then morpholine (0.04 mL, 0.5 mmol) was added and the reaction mixture was stirred at RT for 16 h. The reaction mixture was diluted with EtOAc and washed with 5% aq. Na₂CO₃, water and brine. It was dried over Na₂SO₄, filtered and evaporated to yield brown gum. The crude was purified by RP prep HPLC purification and lyophilized to yield 1-morpholino-2-((4-(o-tolyl)quinolin-7-yl)amino)propan-1-one (10 mg). LCMS (ESI): calculated for C₂₃H₂₅N₃O₂, 375, found [M+H]⁺: 376; ¹H NMR (400 MHz, DMSO-d₆) δ 8.67 (d, 1H), 7.38-7.30 (m, 3H), 7.18-7.16 (m, 1H), 7.05 (br s, 2H), 6.95 (d, 1H), 6.89 (d, 1H), 6.49 (d, 1H), 4.69-4.65 (m, 1H), 3.77-3.73 (m, 1H), 3.65-3.54 (m, 6H), 3.44-3.40 (m, 1H), 1.98 (s, 3H), 1.32 (d, 3H).

Synthesis of N,N-dimethyl-2-((4-(o-tolyl)quinolin-7-yl)amino)propanamide [Step 5]: To a stirred solution of (4-(o-tolyl)quinolin-7-yl)alanine (100 mg, 0.3 mmol) in DMF (1 mL), HATU (250 mg, 0.7 mmol), and N,N-Diisopropylethylamine (0.3 mL, 1.6 mmol) were added at 0° C. and the reaction mixture was stirred for 30 min. Then N-methylmethanamine (20 mg, 0.5 mmol) was added and the reaction mixture was stirred at RT for 16 h. The reaction mixture was diluted with EtOAc and washed with 5% aq. Na₂CO₃, water and brine. It was dried over Na₂SO₄, filtered and evaporated to yield brown gum. The crude was purified by RP prep HPLC purification and lyophilized to yield N,N-dimethyl-2-((4-(o-tolyl)quinolin-7-yl)amino)propanamide (15 mg). LCMS (ESI): calculated for C₂₁H₂₃N₃O, 333, found [M+H]⁺: 334; ¹H NMR (400 MHz, DMSO-d₆) δ 8.65 (d, 1H), 7.40-7.7.37 (m, 2H), 7.34-7.30 (m, 1H), 7.18-7.15 (m, 1H), 7.05 (d, 2H), 6.94 (d, 1H), 6.83 (d, 1H), 6.46 (d, 1H), 4.63-4.60 (m, 1H), 3.17 (d, 3H), 2.87 (br s, 3H), 1.98 (s, 3H), 1.32 (d, 3H).

### Example 48-49

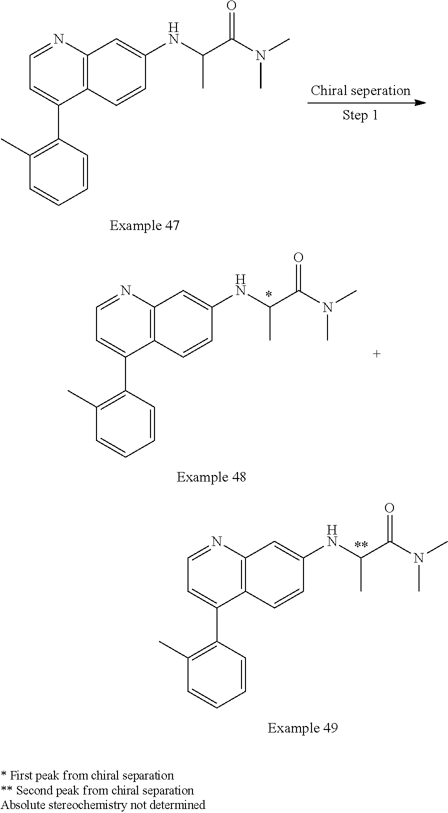

Synthesis of chiral analogs of N,N-dimethyl-2-((4-(o-tolyl)quinolin-7-yl)amino)propanamide [Step 1]: 40 mg of N,N-dimethyl-2-((4-(o-tolyl)quinolin-7-yl)amino)propanamide was further purified via chiral HPLC (SFC) purification and lyophilized to afford Example 48, Peak 1, N,N-dimethyl-2-((4-(o-tolyl)quinolin-7-yl)amino)propanamide (5.0 mg) and Example 49, Peak 2, as N,N-dimethyl-2-((4-(o-tolyl)quinolin-7-yl)amino)propanamide (9.0 mg). The absolute stereochemistry of both enantiomers was not determined.

Example 48, Peak 1: N,N-dimethyl-2-((4-(o-tolyl)quinolin-7-yl)amino)propanamide: LCMS (ESI) Calcd. For C₂₁H₂₃N₃O: 333, found [M+H]⁺=334; ¹H NMR (400 MHz, DMSO-d₆) δ 8.65 (d, 1H), 7.40-7.37 (m, 2H), 7.34-7.30 (m, 1H), 7.18-7.15 (m, 1H), 7.05 (d, 2H), 6.94 (d, 1H), 6.83 (d, 1H), 6.46 (d, 1H), 4.63-4.60 (m, 1H), 3.17 (d, 3H), 2.87 (br s, 3H), 1.98 (s, 3H), 1.32 (d, 3H).

Example 49, Peak 2: N,N-dimethyl-2-((4-(o-tolyl)quinolin-7-yl)amino)propanamide: LCMS (ESI) Calcd. For C₂₁H₂₃N₃O: 333, found [M+H]⁺=334; ¹H NMR (400 MHz, DMSO-d₆) δ 8.65 (d, 1H), 7.40-7.37 (m, 2H), 7.34-7.30 (m, 1H), 7.18-7.15 (m, 1H), 7.05 (d, 2H), 6.94 (d, 1H), 6.83 (d, 1H), 6.46 (d, 1H), 4.63-4.60 (m, 1H), 3.17 (d, 3H), 2.87 (br s, 3H), 1.98 (s, 3H), 1.32 (d, 3H).

Prep HPLC (SFC) method: Column: (R,R) WHELK-01 (21 mm×250 mm), 5p. Flow: 50 g/min. Mobile Phase: 60% CO2+40% (0.1% Triethylamine in Methanol/ethyl acetate (90/10)). ABPR: 100 bar. Temp: 35° C. UV: 254 nm. Diluent: Methanol & Acetonitrile. Sample concentration: 5 mg/ml. Loading. 3 mg/4 min.

### Example 50-51

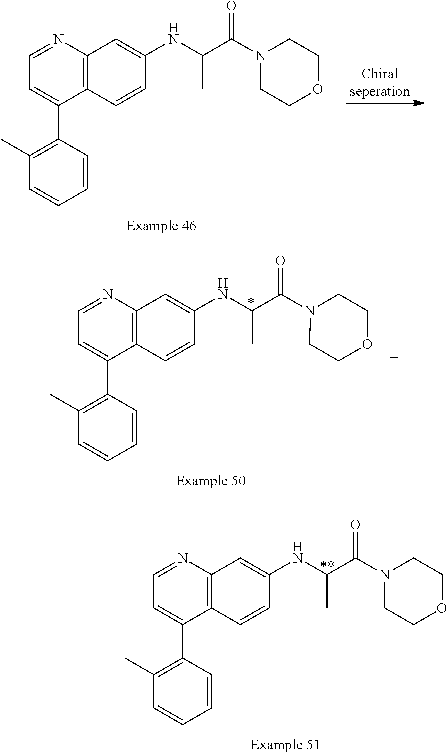 [SMILES: Cc1ccccc1-c1ccnc2cc(NC(C)C(=O)N3CCOCC3)ccc12 ; Cc1ccccc1-c1ccnc2cc(NC(C)C(=O)N3CCOCC3)ccc12 ; Cc1ccccc1-c1ccnc2cc(NC(C)C(=O)N3CCOCC3)ccc12]

Synthesis of chiral analogs of 1-morpholino-2-((4-(o-tolyl)quinolin-7-yl)amino)propan-1-one: 35 mg of 1-morpholino-2-((4-(o-tolyl)quinolin-7-yl)amino)propan-1-one was submitted for NP chiral HPLC purification. The fractions obtained from prep are lyophilized to obtain Example 50, Peak 1, as 1-morpholino-2-((4-(o-tolyl)quinolin-7-yl)amino)propan-1-one (15 mg) and Example 51, Peak 2, as 1-morpholino-2-((4-(o-tolyl)quinolin-7-yl)amino)propan-1-one (10 mg).

Example 50, Peak 1: 1-morpholino-2-((4-(o-tolyl)quinolin-7-yl)amino)propan-1-one: LCMS (ESI). calculated for C₂₃H₂₅N₃O₂, 375, found [M+H]⁺: 376; ¹H NMR (400 MHz, DMSO-d₆) δ 8.67 (d, 1H), 7.39-7.30 (m, 3H), 7.18-7.16 (m, 1H), 7.05 (br s, 2H), 6.95 (d, 1H), 6.89 (d, 1H), 6.49 (d, 1H), 4.69-4.65 (m, 1H), 3.79-3.73 (m, 1H), 3.65-3.64 (m, 6H), 3.44-3.40 (m, 1H), 1.98 (s, 3H), 1.32 (d, 3H).

Example 51, Peak 2: 1-morpholino-2-((4-(o-tolyl)quinolin-7-yl)amino)propan-1-one: LCMS (ESI): calculated for C₂₃H₂₅N₃O₂, 375, found [M+H]⁺: 376; ¹H NMR (400 MHz, DMSO-d₆) δ 8.67 (d, 1H), 7.39-7.30 (m, 3H), 7.18-7.16 (m, 1H), 7.05 (br s, 2H), 6.95 (d, 1H), 6.89 (d, 1H), 6.49 (d, 1H), 4.69-4.65 (m, 1H), 3.79-3.73 (m, 1H), 3.65-3.64 (m, 6H), 3.44-3.40 (m, 1H), 1.98 (s, 3H), 1.32 (d, 3H). The stereochemistry of both the isomers was not determined.

Chiral prep HPLC method: CHIRALPAK AY-H (250×21 mm) 5u, Flow rate—21 ml/min, Mobile phase—HEXANE/ETOH/IP AMINE—70/30/0.1, Solubility—MEOH Wave length—258 nm, Run time—23 min.

### Example 52-53

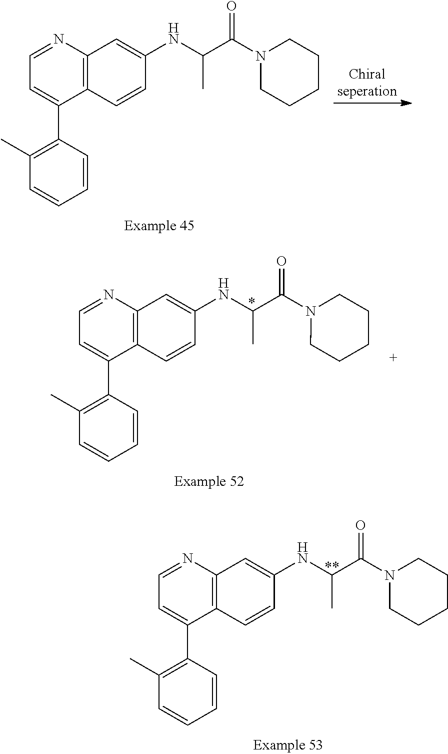 [SMILES: Cc1ccccc1-c1ccnc2cc(NC(C)C(=O)N3CCCCC3)ccc12 ; Cc1ccccc1-c1ccnc2cc(NC(C)C(=O)N3CCCCC3)ccc12 ; Cc1ccccc1-c1ccnc2cc(NC(C)C(=O)N3CCCCC3)ccc12]

Synthesis of chiral analogs of 1-(piperidin-1-yl)-2-((4-(o-tolyl)quinolin-7-yl)amino)propan-1-one: 35 mg of 1-(piperidin-1-yl)-2-((4-(n-tolyl)quinolin-7-yl)amino)propan-1-one was submitted for chiral HPLC SFC purification. The fractions obtained from prep are lyophilized to obtain Example 52, Peak 1, as 1-(piperidin-1-yl)-2-((4-(o-tolyl)quinolin-7-yl)amino)propan-1-one (10 mg) and Example 53, Peak 2, as 1-(piperidin-1-yl)-2-((4-(o-tolyl)quinolin-7-yl)amino)propan-1-one (20 mg).

Example 52, Peak 1: 1-(piperidin-1-yl)-2-((4-(o-tolyl)quinolin-7-yl)amino)propan-1-one: LCMS (ESI): calculated for C₂₄H₂₇N₃O: 373, found [M+H]⁺: 374: ¹H NMR (400 MHz, DMSO-d₆) δ 8.76 (d, 1H), 7.47-7.41 (m, 2H), 7.38-7.34 (m, I H), 7.23-7.19 (m, 5H), 6.84 (d, 1H), 4.67-4.63 (m, 1H), 3.64-3.41 (m, 4H), 2.01 (s, 3H), 1.64 (br s, 3H), 1.56 (br s, 1H), 1.45 (br s, 2H), 1.35 (d, 3H).

Example 53, Peak 2: 1-(piperidin-1-yl)-2-((4-(o-tolyl)quinolin-7-yl)amino)propan-1-one: LCMS (ESI): calculated for C₂₄H₂₇N₃O: 373, found [M+H]⁺: 374; ¹H NMR (400 MHz, DMSO-d₆) δ 8.76 (d, 1H), 7.47-7.41 (m, 2H), 7.38-7.34 (m, 1H), 7.23-7.19 (m, 5H), 6.84 (d, 1H), 4.67-4.63 (m, 1H), 3.64-3.41 (m, 4H), 2.01 (s, 3H), 1.64 (br s, 3H), 1.56 (br s, 1H), 1.45 (br s, 2H), 1.35 (d, 3H). The stereochemistry of both the isomers is not determined.

Chiral prep HPLC method: Chiral HPLC method: Column: (R,R) WHELK-01 (21.1 mm×250 mm), 5μ Flow: 60 g/min Mobile Phase: 60% CO2+40% (0.3% Ipamine MEOH) ABPR: 100 bar Temp: 35° C. UV: 210 nm DILUENT: MeOH+THF SAMPLE CONCENTRATION: 13.3 mg/ml loading:5.3 mg/4.5 min.

### Example 54

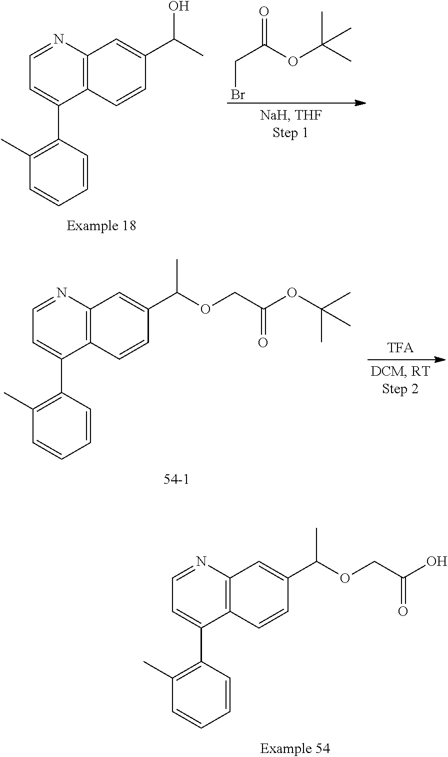

Synthesis of tert-butyl 2-(1-(4-(o-tolyl)quinolin-7-yl)ethoxy)acetate [Step 1]: To a stirred solution of 1-(4-(o-tolyl)quinolin-7-yl)ethan-1-ol (600 mg, 2.3 mmol) in THF (20 mL)) was added NaH (82 mg, 3.42 mmol, 60% in oil) at 0° C. and stirred for 15 min. After that tert-butyl 2-bromoacetate (533 mg, 2.7 mmol) was added to it under ice cold condition and stirred at Rt for 16 h. Reaction mass was quenched with saturated NH4Cl solution and extracted with EtOAc. Organic part was evaporated to get crude. Crude material was purified by combi-flash to afforded tert-butyl 2-(1-(4-(o-tolyl)quinolin-7-yl)ethoxy)acetate (500 mg). Crude material was used for next step without further purification. LCMS (ESI) Calcd. for C₂₄H₂₇NO₃: 377, found [M+H]⁺=378.

Synthesis of 2-(1-(4-(o-tolyl)quinolin-7-yl)ethoxy)acetic acid [Step 2]: To a stirred solution of tert-butyl 2-(1-(4-(o-tolyl)quinolin-7-yl)ethoxy)acetate (80 mg, 0.2 mmol) in DCM (5 mL) was added TFA (0.024 mL, 0.32 mmol) at 0° C. and stirred at rt for 48 h. Reaction mixture was concentrated in vacuo. The crude was purified by prep HPLC purification and lyophilized to afford 2-(1-(4-(o-tolyl)quinolin-7-yl)ethoxy)acetic acid (30 mg). LCMS (ESI) Calcd. for C20H19NO3: 321, found [M+H]+=322; ¹H NMR (400 MHz, DMSO-d₆): δ 8.95-8.94 (d, 1H), 7.99 (s, 1H), 7.54-7.52 (d, 1H), 7.43-7.33 (m, 5H), 7.24-7.21 (m, 1H), 4.83-4.78 (t, 1H), 3.88-3.73 (m, 2H), 1.98 (s, 3H), 1.45-1.43 (s, 3H).

### Example 55

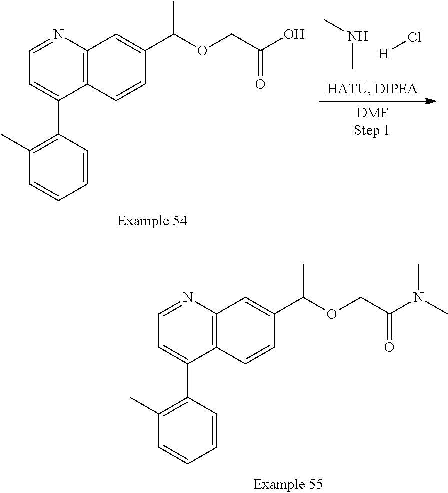

Synthesis of N,N-dimethyl-2-(1-(4-(o-tolyl)quinolin-7-yl)ethoxy)acetamide [Step 1]: To a stirred solution of 2-(1-(4-(o-tolyl)quinolin-7-yl)ethoxy)acetic acid (150 mg, 0.47 mmol) and dimethyl amine hydrochloride (95 mg, 1.2 mmol) in DMF (1 mL), was added DIPEA (0.25 mL, 1.9 mmol) followed by HATU (532 mg, 1.40 mmol) at 0° C. and stirred at room temperature for 16 h. Reaction mixture was diluted with EtOAc and washed with water and brine, dried over anhydrous sodium sulfate and concentrated in vacuo. The crude was purified by prep HPLC purification and lyophilized to afford N,N-dimethyl-2-(1-(4-(o-tolyl)quinolin-7-yl)ethoxy)acetamide (25 mg). LCMS (ESI) Calcd. for C₂₂H₂₄N₂O₂: 348, found [M+H]+=349; ¹H NMR (400 MHz, DMSO-d₆): δ 8.95-8.94 (d, 1H), 8.0 (s, 1H), 7.55-7.53 (d, 1H), 7.44-7.35 (m, 5H), 7.24 (d, 1H), 4.80-4.78 (t, 1H), 4.09-4.08 (t, 2H), 2.86 (s, 6H), 2.10 (s, 3H), 1.52-1.50 (d, 3H).

### Example 56

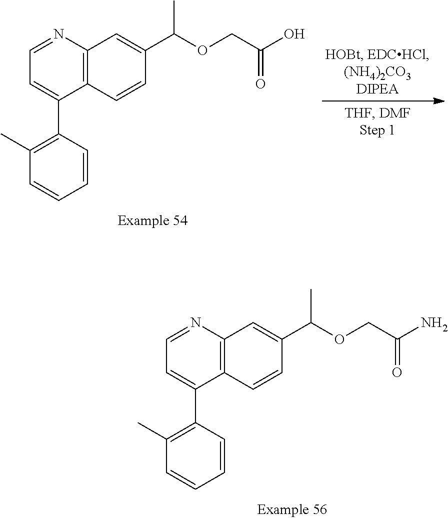 [SMILES: Cc1ccccc1-c1ccnc2cc(C(C)OCC(=O)O)ccc12 ; Cc1ccccc1-c1ccnc2cc(C(C)OCC(N)=O)ccc12]

Synthesis of 2-(1-(4-(o-tolyl)quinolin-7-yl)ethoxy)acetamide [step-1]: To a stirred solution of 2-(1-(4-(o-tolyl)quinolin-7-yl)ethoxy)acetic acid (150 mg, 0.47 mmol) in THF (3 mL) and DMF (0.8 mL) were added HOBt (95 mg, 0.7 mmol), EDC·HCl (134 mg, 0.7 mmol) and DIPEA (0.25 mL, 1.87 mmol) followed by (NH₄)₂CO₃ (179 mg, 1.87 mmol) and stirred at RT for 16 h. The Reaction mixture was diluted with water and extracted with EtOAc. The organic layer was washed with brine, dried over anhy. Na₂SO₄, concentrated to get crude. The crude was purified by prep HPLC purification and lyophilized to 2-(1-(4-(o-tolyl)quinolin-7-yl)ethoxy)acetamide (30 mg). LCMS (ESI) Calcd. for C20H20N2O2: 320, found [M+H]+=321; ¹H NMR (400 MHz, DMSO-d₆) δ 8.96-8.95 (d, 1H), 8.04-8.03 (s, 1H), 7.58-7.56 (d, 1H), 7.43-7.35 (m, 5H), 7.26 (m, 3H), 4.76-4.74 (t, 1H), 3.75-3.69 (t, 2H), 1.99-1.98 (s, 3H), 1.50-1.48 (d, 3H).

### Example 57

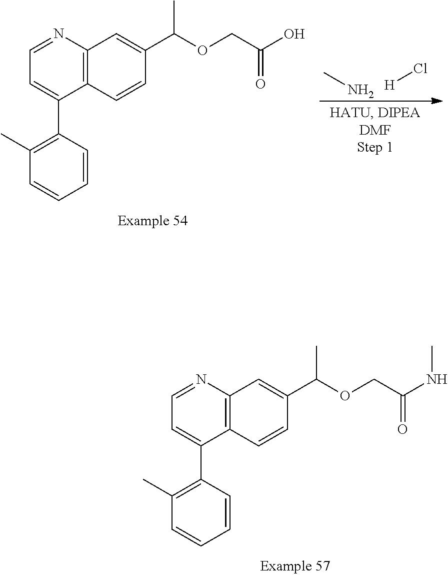

Synthesis of N-methyl-2-(1-(4-(o-tolyl)quinolin-7-yl)ethoxy)acetamide [Step 1]: To a stirred solution of 2-(1-(4-(o-tolyl)quinolin-7-yl)ethoxy)acetic acid (150 mg, 0.47 mmol) and methanamine hydrochloride (47 mg, 0.700 mmol) in DMF (3 mL), was added DIPEA (0.25 mL, 1.9 mmol) followed by HATU (532 mg, 1.40 mmol) at 0° C. and stirred at rt for 16 h. Reaction mixture was diluted with EtOAc and washed with water and brine, dried over anhydrous sodium sulfate and concentrated in vacuo. The crude was purified by prep HPLC purification and lyophilized to afford N-methyl-2-(1-(4-(o-tolyl)quinolin-7-yl)ethoxy)acetamide (18 mg). LCMS (ESI) Calcd. for C21H22N2O2: 334, found [M+H]+=335; ¹H NMR (400 MHz, DMSO-d₆): δ 8.96-8.94 (d, 1H), 8.04 (s, 1H), 7.73 (br s, 1H), 7.57-7.55 (d, 1H), 7.42-7.33 (m, 5H), 7.23 (d, 1H), 4.77-4.73 (t, 1H), 3.80-3.77 (d, 1H), 3.73-3.68 (m, 1H), 2.62-2.61 (d, 3H), 1.98 (s, 3H), 1.50-1.49 (d, 3H).

### Biological/Biochemical Evaluation

### General Protocol for In Vitro Analysis of Compounds

The inhibitory activity of the compounds of the present invention against POLRMT were determined by assays based on Bergbrede, T., et al., “An adaptable high-throughput technology enabling the identification of specific transcription modulators,” SLAC Discov., 22, 378-386 (2017).

The ability of some compounds of the present invention to inhibit POLRMT were determined in a homogeneous TR-FRET Assay using high-throughput screening in a 384-well plate format. This method is used to monitor the activity of mitochondrial transcription through measurement of its product, a 407 bp long RNA transcript. Detection of the product is facilitated by hybridization of two DNA-oligonucleotide probes to specific and adjacent sequences within the RNA product sequence. Upon annealing of the probes, two fluorophores are coupled directly to an acceptor nucleotide probe (ATTO647, 5′), or introduced via a coupled streptavidin with a biotinylated donor nucleotide probe (Europium cryptate) that is brought into sufficient proximity to serve as a fluorescence-donor-acceptor pair. Thus, a FRET signal at 665 nm is generated upon excitation at 340 nm.

Proteins used as transcription factors (POLRMT: NP_005026.3, TFAM: NP_003192.1, TFB2M: NP_071761.1) are diluted from their stocks to working concentrations of 1 μM, 20 μM and 4 μM respectively, in a dilution buffer containing 20 mM Tris-HCl (pH 8.0), 200 mM NaCl, 10% (v/v) glycerol, 1 mM Dithiothreitol (DTT), 0.5 mM EDTA.

DNA template is a pUC18 plasmid with the mitochondrial light strand promotor sequence (1-477) cloned between HindIII and BamHI sites. The DNA template is restriction linearized proximal to the promotor 3′-end (pUC-LSP).

The reaction mixture (10 uL) containing 7.5 nM POLRMT, 15 nM of TFB2M, 30 nM of TFAM, 0.5 nM of DNA template and 500 μM nucleotide triphosphate mix (NTPs) in a reaction buffer (containing 10 mM Tris-HCl (pH 7.5), 10 mM MgCl₂, 40 mM NaCl, 10 mM DTT, 0.005% (w/v) Tween-20, 160 units/ml Rnase inhibitor and 0.1 mg/mL BSA) are dispensed to compounds in microplates, using a Thermo Multidrop® dispenser, and incubated at 37° C. in a VWR INCU-Line incubator for 60 minutes after mixing. No nucleotide triphosphate mix is added to negative control samples. Microplates with compounds to be tested in the assay are prepared from 10 mM compound stocks in 100% DMSO, equal amounts of DMSO without any compound are added to positive control and negative control samples.

During the incubation, a mix of the detection reagents is prepared in a buffer such that the enzymatic reaction is terminated due to chelating of Mg-ions and increased ionic strength, containing 50 mM Tris-HCl (pH 7.5), 700 mM NaCl, 20 mM EDTA, and 0.01% (w/v) Tween-20. Europium-streptavidin is pre-incubated with a 200-fold molar excess of a random sequence oligonucleotide to block unspecific binding of oligo, for two hours at ambient temperature in the dark. Afterwards, the blocked Europium-streptavidin is kept on ice until use.

At the end of the enzymatic reaction time, 5 μL detection oligo mix in the detection buffer is added, and assay plates are mixed and kept at ambient temperature for one hour, protected from light. The concentration of the Acceptor nucleotide oligo (e.g., ATTO647N-5′-ACAAAGAACCCTAACACCAG-3′ (SEQ ID NO: 1)) and Donor nucleotide oligo (e.g., bio-5′-AACACATCTCT(-bio)GCCAAACCCCA-bio-3′ (SEQ ID NO: 2)) in each assay well is 1 nM, and 3 nM, respectively.

After incubation with oligo mix, 5 μL of pre-blocked Europium-streptavidin reagent is dispensed to each assay well, assay plates are again mixed and kept at ambient temperature for one hour, protected from light.

The generated signal is measured with BMG Pherastar microtiter plate reader with a TRF light unit, using excitation at 340 nm, an integration time of 200 μs, and a delay time of 100 ps, before detection at 620 nm and 665 nm. The ratio of donor- and acceptor-fluorescence is used as a measure of the generated transcript product (i.e. enzymatic activity).

The IC₅₀ values are summarized in Table 1.

TABLE 1
IC₅₀ values for isoquinolinone compounds.

| Examples | IC₅₀ (μM) |
|---|---|
| Example 1 | 3 |
| Example 2 | >100 |
| Example 3 | 26 |
| Example 4 | >100 |
| Example 5 | >100 |
| Example 6 | 90 |
| Example 7 | >100 |
| Example 8 | >100 |
| Example 9 | >100 |
| Example 10 | 50 |
| Example 11 | 10 |
| Example 13 | 29 |
| Example 15 | 59 |
| Example 12 | 15 |
| Example 16 | 7 |
| Example 17 | 10 |
| Example 14 | 68 |
| Example 19 | 5 |
| Example 20 | >100 |
| Example 21 | >100 |
| Example 22 | 72 |
| Example 25 | >100 |
| Example 18 | 36 |
| Example 24 | 10 |
| Example 23 | >100 |
| Example 26 | >100 |
| Example 27 | 8 |
| Example 28 | >100 |
| Example 31 | 6 |
| Example 32 | 24 |
| Example 29 | 97 |
| Example 30 | 91 |
| Example 33 | 74 |
| Example 34 | 2 |
| Example 35 | 70 |
| Example 36 | >100 |
| Example 37 | 79 |
| Example 38 | 37 |
| Example 39 | >100 |
| Example 40 | >100 |
| Example 41 | 91 |
| Example 42 | >100 |
| Example 43 | >100 |
| Example 44 | >100 |
| Example 45 | 4 |
| Example 46 | 8 |
| Example 47 | 26 |
| Example 48 | >100 |
| Example 49 | 10 |
| Example 50 | 2 |
| Example 51 | >100 |
| Example 52 | >100 |
| Example 53 | 3 |
| Example 54 | >100 |
| Example 55 | >100 |
| Example 56 | >100 |
| Example 57 | >100 |

### General Protocol for In Vivo AML (Acute Myeloid Leukemia) Efficacy Experiment

### Determination of Maximum Tolerated Dose of Test Compound

Immunocompromised mice (6-10-week-old, female NSG mice, strain NOD.Cg-Prkdc^{scid} Il2rg^{tm1Wjl}/Szj, Jackson Laboratories) are treated orally with test compound ranging from 75 to 150 mg/kg, once or twice per day for the duration of 14 days. Total body weight is measured, and the general condition of mice is monitored routinely. Mice with severe symptoms and moribund are excluded from study. Submental blood collection method (no anesthesia) is used for all samplings. Plasma levels of test compound are determined at intervals ranging from 0.5 to 4 hours post first and last doses in all dosing groups. From these data pharmacokinetic analysis are conducted.

### In Vivo Efficacy Study in AML Mouse Model

MV4-11 AML cell lines (ATCC) are labelled with luciferase tag by viral transduction procedure (MV4-11-luc).

For an AML cell line xenograft efficacy experiment, female NSG mice are given intravenously ˜1×10⁷ MV4-11-luc cells. Mice are flux sorted and randomized into treatment groups 14 days post transplantation. Mice are then treated with vehicle (50 mM Na₂HPO₄), or test compound at a tolerable dose determined from the above study, once or twice per day for 21 days. Tumor progression/regression is monitored by imaging of mice using luciferin as a substrate (150 mg/kg). Images are taken on a total of 9 time points i.e., one flux sort and once weekly to end date (8 time points). Imaging is performed under anesthesia and using in vivo imaging equipment IVIS. The treatment efficacy is also measured based on proportion of human AML cells, determined by flow cytometry analysis of viable human CD45 positive cell population in peripheral blood of mice one week post last dose. Plasma levels of test compound are determined at intervals ranging from 0.5 to 4 hours post last dose. Animals are monitored individually, and total body weight is measured routinely. The endpoint of the experiment is moribundity. In addition, mice demonstrating tumor-associated symptoms including impairment of hind limb function, ocular proptosis, and weight loss are considered for euthanasia. The remaining mice are euthanized on day 60 of the study.

1. A compound, a prodrug thereof, or a pharmaceutically acceptable salt thereof, represented by formula (I): 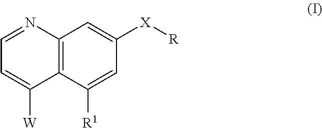
wherein:
X is —C₁-C₆ alkyl-, —N(R³)—, —C(O)—;
W is C₆-C₁₀ aryl, C₆-C₁₀ cycloaryl, or 5-10 membered heteroaryl, wherein the aryl, cycloaryl, or heteroaryl are each optionally substituted with one or more groups independently selected from the group consisting of fluoro, chloro, C₁-C₄ alkyl, trifluoromethyl, difluoromethyl, cyano, hydroxyl, and C₁-C₄ alkoxyl;
R is hydrogen, C₁-C₆ alkyl, hydroxy, NR²R³, acyl, carboxy, C(O)NR²R³, or NR⁴—S(O)₂R² wherein the alkyl group is optionally substituted with one or more groups each independently selected from the group consisting of hydroxyl, cyano, fluoro, C₁-C₄ alkoxyl, C₁-C₄ haloalkoxyl, aryl, 5- or 6-membered heterocyclyl, C(O)OR⁴, NR²R³, and C(O)NR²R³,
or R is C₁-C₄ alkoxyl optionally substituted with hydroxyl, fluoro, NR²R³, carboxyl, and C(O)NR²R³,
or R is C₃-C₆ cycloalkyl, or 5- or 6-membered heterocyclyl, wherein the cycloalkyl and heterocyclyl are each optionally substituted with oxo or C₁-C₄ alkyl,
or R is C₆-C₁₀ aryl or C₅-C₆ heteroaryl containing one or more heteroatoms that is N, O, or S, wherein the aryl and heteroaryl are optionally substituted with one or more groups each independently selected from the group consisting of fluoro, chloro, C₁-C₄ alkyl, trifluoromethyl, difluoromethyl, cyano, hydroxyl, C₁-C₄ haloalkoxyl, C₁-C₄ alkoxyl, and acyl;
R¹ is hydrogen, fluoro, chloro, hydroxyl, cyano, or C₁-C₃ alkyl optionally substituted with one or more fluorines, C₃-C₄ cycloalkyl, C₁-C₂ alkoxyl or C₁-C₂ haloalkoxyl;
each R² is independently hydrogen, C₁-C₄ alkyl, C₃-C₆ cycloalkyl, hydroxyl, or aryl, wherein the alkyl, cycloalkyl, and aryl are optionally substituted with one or more groups selected from the group consisting of C₃-C₆ cycloalkyl, fluoro, hydroxyl, C₁-C₄ alkoxyl, C₁-C₄ haloalkoxyl, aryl, carboxyl, C(O)NR⁴R⁴;
each R³ is independently R², C(O)C₁-C₄ alkyl, C(O)C₃-C₆ cycloalkyl, C(O)C₆-C₁₀ aryl, C(O)-5- to 10-membered heteroaryl, C(O)O—C₁-C₄ alkyl, or C(O)NHC₁-C₄ alkyl, wherein each alkyl, cycloalkyl, aryl, and heteroaryl are optionally substituted with one or more groups selected from the group consisting of C₁-C₄ alkyl, fluoro, chloro, trifluoromethyl, difluoromethyl, cyano, hydroxyl, and C₁-C₄ alkoxyl,
or if R² and R³ are attached to the same nitrogen atom, R² and R³ together with their connecting nitrogen can form a 5- or 6-membered heterocyclic ring optionally containing another heteroatom that is N, O, or S, and optionally substituted with one or two groups each independently selected from the group consisting of fluoro, chloro, C₁-C₄ alkyl, C₁-C₄alkoxyl, C₁-C₄ haloalkoxyl, acyl, carboxy, C₁-C₄ alkyl carboxy, and C₁-C₄ alkylcarboxylate; and
R⁴ is hydrogen or C₁-C₄ alkyl.


SEQUENCE LISTING (2 sequences)

SEQ ID NO: 1 - DNA, 20 bp, synthetic construct
  source 1..20: mol_type = other DNA
acaaagaaccctaacaccag

SEQ ID NO: 2 - DNA, 22 bp, synthetic construct
  source 1..22: mol_type = other DNA
  modified_base 11: mod_base = OTHER; note = Biotin modified nucleotide
aacacatctctgccaaacccca